(19) United States
(12) Patent Application Publication
(10) Pub. No.: US 2009/0304750 A1
(43) Pub. Date: Dec. 10, 2009

(54) NOVEL PRIME-BOOST COMBINATIONS OF ATTENUATED MYCOBACTERIUM

(76) Inventors: David Hone, Rockville, MD (US); Jerald C. Sadoff, Washington, DC (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 12/097,146

(22) PCT Filed: Dec. 15, 2006
(86) PCT No.: PCT/US06/62143

§ 371 Date: Feb. 21, 2009

Related U.S. Application Data
(60) Provisional application No. 60/750,348, filed on Dec. 15, 2005.

(51) Int. Cl. A61K 39/04; A61K 39/112; A61K 31/7088; A61P 37/04

(52) U.S. Cl. 424/248.1; 514/44 R

(57) ABSTRACT

The present invention provides vaccine compositions for effective induction of both mucosal and systemic immunity to pathogenic Mycobacterium species. Vaccination protocols are provided in which both parenteral and mucosal vaccine formulations are administered to a host. The parenteral and mucosal formulations comprise live, attenuated Mycobacteria.

### FIELD OF THE INVENTION

The invention generally relates to methods for vaccinating a host against pathogenic Mycobacterium species. In particular, the invention provides a vaccine protocol in which both parenteral and mucosal formulations of live, attenuated Mycobacteria are administered sequentially to the host, resulting in both systemic and mucosal immune responses to the live, attenuated Mycobacteria.

### BACKGROUND OF THE INVENTION

Tuberculosis (TB) is an enormous and deadly problem in the developing world, killing millions of people in the prime of their lives every year. It is a leading cause of death in HIV-infected individuals (11, 15, 16, 43, 44) and in women of childbearing age (61, 63, 65). The World Health Organization (WHO) estimates that each year there are 8 million new cases of TB and 2 million deaths due to TB (5, 24). Among infectious diseases, only HIV and diarrheal diseases kill more people.

In 1993, the WHO designated TB a global public health emergency (1, 3). Ninety-nine percent of the estimated 2 million TB deaths and 95% of the 8 million new cases each year occur in low and middle-income countries comprising 85% of the world's population (5, 11, 15, 16, 24, 43, 44). Despite widespread use of DOTs (Directly-Observed Therapy Short-course) and Bacille Calmette-Guerin (BCG), TB is now a leading cause of severe disease and death in the developing world (2, 26, 59). The uncontrolled TB epidemic has been exacerbated in developing countries by many causes including pandemic HIV, war and political instability, drug resistance, and increasing poverty (5, 11, 15, 16, 24, 43, 44).

Although TB can be treated with drugs, the basic therapeutic regimen requires at least six months to complete and as many as four different drugs need to be taken. In combination with drug therapy, a moderately effective vaccine against TB could substantially reduce the disease burden. The currently licensed TB vaccine, BCG, has been in use since early in the 20th century and is administered to millions of newborns around the world; it is thought to be effective in the first few years of life against severe TB disease. However, the fact the TB epidemic remains unchecked (2, 26, 59) illustrates the urgent need for a better TB vaccine.

Through the application of genomics (17-19) and proteomics (4, 22, 51, 58, 64, 66) a number of strategies have emerged to improve protection against TB through vaccination. These strategies can be placed into three major categories.

### Category 1. Modified Recombinant BCG or Live Attenuated Mtb

This category is based on the idea that BCG is modestly effective and can form the basis of an improved TB vaccine. Three general approaches have been developed to improve BCG. The first approach, developed initially by Horwitz and coworkers (33, 35), entails expanding the repertoire of immunogenic antigens in BCG. Thus, increased expression of Rv1886c (also known as “antigen 85B”) in BCG improved the protective properties of BCG Tice (33, 35). This observation is in agreement with reports by others showing the rBCG strains that express an expanded antigen repertoire afford better protection in laboratory animals than the respective parental BCG strains (40, 50, 53).

The second approach is based on the idea that modifications of the host-BCG interaction will improve protection afforded by the resulting rBCG. Examples of this approach include rBCG strains with modified sodA expression (25) and strains that are engineered to escape the endosome (29, 31). In both instances, the resulting strains are believed to augment antigen trafficking via cross presentation pathways, thereby invoking enhanced immune responses to the vaccine antigens (25, 29). In addition, these strategies improved protection against a low-dose aerosol challenge in mice (25). The third approach in this category is based on the idea that BCG, a derivative of Mycobacterium bovis, does not present the full set of antigens expressed by Mycobacterium tuberculosis (Mtb, the causative agent of TB) during infection, and those antigens that the two bacteria have in common display some allelic polymorphism. Thus, it has been argued that use of an attenuated Mtb, which would present the identical set of genes as those expressed in Mtb-infected individuals, would be preferable to the use of BCG (32, 56, 57). Although this approach has yet to produce a TB vaccine that displays greater protection than BCG in animal models, attenuated Mtb have proven safer than BCG in animal models of immunodeficiency (32, 56, 57).

### Category 2. Subunit and Vector Vaccines

The second strategy stems from the observation that certain TB proteins when administered as subunit vaccines appear to invoke protective immunity in animal models. Among the antigens that induce protection, ESAT-6 and the so-called antigen 85 complex have received the lion's share of attention (12, 36, 37, 42, 47-49, 62, 67). More recently, it has become evident that some fusion proteins comprised of two or more candidate TB vaccine antigens are more effective than the individual components. Lead candidates that fall into this subcategory are Hybrid-1, a fusion protein comprised of ESAT6 and Rv1886c (42, 48); Hyvac-4, a fusion protein comprised of Rv0288 and Rv1886c (21); and 72f, a fusion protein comprised of Rv125 and Rv1196 (14, 38, 60). These fusion proteins, when formulated with an appropriate adjuvant, have proven effective at affording protection in animal models (12, 14, 21, 37, 38, 42, 47, 48, 60, 62, 67).

### Category 3. Heterologous Prime-Boost Vaccine Regimens

The above-mentioned strategies rely on individual vaccine modes, either given as a single-dose or in prime-boost regimens, with the goal of inducing long-lived potent Mtb-specific immunity. However, it is widely acknowledged that two doses of BCG or attenuated Mtb do not improve efficacy over that afforded by a single dose of these vaccines (54), despite being safe and more immunogenic than a single dose of BCG (6, 23). Moreover, multiple doses of either subunit or viral vector vaccines may be too expensive to be of practical use in the developing world where TB is prevalent.

Accordingly, a third strategy has gained attention recently in which a heterologous booster vaccine is utilized to bolster immunity elicited by the prime. Indeed, BCG-primed individuals develop impressive cellular immune responses following a heterologous boost comprised of modified vaccinia Ankara (MVA) encoding Mtb antigen 85A (herein “Ag85A”; also known as Rv3804c; (28, 45, 46); in contrast, naive individuals develop relatively unimpressive responses to the MVA-Ag85A vaccine (45, 46). In addition, the BCG-prime MVA-Ag85A boost regimen was shown to be more effective than BCG alone at affording protection in mice (28). In addition, heterologous prime-boost regimens that include subunit booster vaccines to boost BCG have also proven more effective than BCG alone (34).

Although the studies cited above did not identify the correlates of protection, when taken as a whole, experimental studies in laboratory animals suggest that heterologous prime-boost regimens are advantageous over single-dose or homologous prime-boost vaccination regimen. Despite these promising developments, however, there continues to be a need to develop vaccination strategies that are affordable to those most in need. Thus, although heterologous prime-boost strategies have proven effective in animal models and merit further evaluation in clinical trials, from a vaccine delivery point of view handling a single vaccine or two forms of the same vaccine is easier than a heterologous prime boost regimen. These vaccine regimens will require cGMP manufacturing, fill, packaging, release, and stability testing of two distinct components, which augments the investment required to move such vaccines forward into large-scale clinical applications, for construction of large scale manufacturing plants and vaccine regimen costs. Furthermore, live attenuated mycobacterial vaccines are inherently cheaper to produce than the booster vaccines currently being considered.

Given the current low level of funding by government, non-profit and corporate organizations, successful control of TB in developing countries by public health vaccine intervention programs may only become a reality when inexpensive prime-boost regimens become available. The prior art has thus far failed to provide such cost effective, efficacious regimens.

### SUMMARY OF THE INVENTION

The present invention provides a novel prime-boost strategy for eliciting an immune response to pathogenic Mycobacterium species. The strategy involves the sequential administration of two different vaccine formulations of live, attenuated Mycobacteria, one of which is formulated for parenteral administration, and the other of which is formulated for mucosal administration. The first formulation that is administered is the “prime” and the second formulation that is administered is the “boost”. The parenteral formulation is designed to elicit primarily a systemic immune response to the antigens in the formulation, whereas the mucosal formulation is designed to elicit primarily a mucosal immune response to the antigens of the live, attenuated Mycobacteria in the formulation. Together, the two immune responses (systemic and mucosal) provide complete, effective protection against infection by and/or the development of disease symptoms caused by Mycobacterium species bearing antigens that are the same or similar to those of the live, attenuated Mycobacteria in the formulations.

The present invention provides a method of eliciting both a systemic and a mucosal immune response to live, attenuated Mycobacteria or to mycobacterial antigens in a host. The method comprises the steps of 1) administering parenterally to said host a first antigenic composition comprising said live, attenuated Mycobacteria, or said mycobacterial antigens, or a vector or bacterium harboring nucleic acids coding for said mycobacterial antigens; and 2) administering mucosally to said host a second antigenic composition comprising said live, attenuated Mycobacteria, or said mycobacterial antigens, or a vector or bacterium harboring nucleic acids coding for said mycobacterial antigens; said second antigenic composition being different from said first antigenic composition. The steps of administering parenterally and administering mucosally result in the induction in said host of both a systemic and a mucosal immune response to said live, attenuated Mycobacteria or said mycobacterial antigens. In one embodiment of the invention, the live, attenuated Mycobacteria is selected from the group consisting of Mycobacterium tuberculosis, Mycobacterium bovis, BCG, Mycobacterium avium complex, M. kansasii, M. malmoense, M. simiae, M. szulgai, M. xenopi, M. scrofulaceum, M. abscessus, M. chelonae, M. haemophilum, M. ulcerans, or M. marinum. In another embodiment of the invention, the bacterium harboring nucleic acids coding for the mycobacterial antigens is a Shigella bacterium. In yet another embodiment, the vector coding for said mycobacterial antigens is an adenoviral vector. In further embodiments of the invention, the live, attenuated Mycobacteria comprise DNA encoding a moiety selected from the group consisting of: a foreign immunogen, an endogenous immunogen, an adjuvant, a cytokine, a pro-apoptosis agent, and an overexpressed Mtb antigen. In one embodiment of the invention, the step of administering mucosally is accomplished orally is carried out as a “prime” i.e. before the “boost” step of administering parenterally.

### BRIEF DESCRIPTION OF THE DRAWINGS

FIG. 1. Schematic flow-chart representation of vaccine manufacturing process.

FIG. 2. The map for suicide vector pAF102. The denotation for each of the DNA segments are as follows: L-flank and R-flank: sequences flanking the 5-prime and 3-prime ends of the ureC gene, respectively; pfoAG137Q encodes the mutant form of perfringolysin O (GenBank Accession no. BA000016) with a single amino acid substitution of glutamine in place of glycine at position 137 (i.e. G137Q); LPPAg85B is the DNA sequence encoding antigen 85B(i.e. Rv1886c) leader peptide; PAg85A is the promoter sequence of antigen 85A gene (i.e. Rv3804c); aph is aminoglycoside phosphotransferase gene (GenBank Accession no. X06402), which confers Kanamycin resistance for the plasmid; OriE is the pUC origin of replication (GenBank Accession no. AY234331); ble encodes Zeocin-resistance (GenBank Accession no. L36850); sacB encodes levansucrase (GenBank Accession no. Y489048), which confers sensitivity to sucrose; Phsp60 is the promoter sequence of heat shock protein gene Rv0440; MCS is the multiple cloning sites for the indicated restriction enzymes. FIG. 3. Map of antigen over-expression vector pAF105. The denotation for each of the DNA segments as follow: PRv3130 the promoter sequence of antigen Rv3130c; PAg85B is the promoter sequence of Rv1886c. The genes in the expression cassette are Rv0288 (10.4); Rv1886c and Rv3804c; aph is aminoglycoside phosphotransferase gene (GenBank Accession no: X06402), which confers kanamycin resistance; oriE is the pUC origin of replication (GenBank Accession no: AY234331); leuD is the gene encoding 3-isopropylmalate dehydratase (i.e. Rv2987c); oriM is the origin of replication in Mycobacterium (GenBank Accession no: M23557).

FIG. 4. Comparison of vaccination with standard BCG vaccine vs a two-component TB vaccine of the present invention.

### DETAILED DESCRIPTION OF THE PREFERRED EMBODIMENTS OF THE INVENTION

The present invention is based on the realization that an optimal strategy for eliciting protective immunity against a pathogenic Mycobacterium species involves the generation of both a systemic and a mucosal immune response to the Mycobacterium species. The invention thus provides a multi-component vaccination method (system, regimen, protocol) in which a first prime dose and a boost dose (or boost doses) differ in their formulations, one being optimized for parenteral administration, and the other for mucosal administration. Both formulations contain live, attenuated Mycobacteria. The parenteral formulation is designed to induce primarily a systemic immune response to the antigens in the formulation, whereas the mucosal formulation is designed to elicit primarily a mucosal immune response to the antigens in the formulation. Upon completion of the administration steps of the system (prime and at least one boost), both systemic and mucosal immune responses develop to the live, attenuated Mycobacteria. The two responses together thus provide complete, effective protection against infection by and/or the development of disease symptoms caused by pathogenic Mycobacterium species which bear the same or similar antigens to those present in the formulations, i.e. to the antigens of the live, attenuated Mycobacteria.

In particular, the present infection provides a method of vaccination a host against Mycobacterium tuberculosis, the causative agent of tuberculosis (TB). Hitherto, there is no prior art describing a two-component TB vaccine comprised of one component formulated for parenteral and another component formulated for mucosal administration. An advantage of the current approach is that this novel combination of vaccine formulations enables the induction of both mucosal and systemic immunity. In previous instances in which live-attenuated Mycobacterium vaccines were used in prime-boost vaccination regimens, the prime and the boost were prepared as identical formulations administered by the same route (6, 23, 54). However, experimental evidence suggests that preexisting immunity to BCG interferes with the boost, resulting in no measurable benefit from the boost compared to the level of protection afforded by the prime alone (13, 20). In contrast, the present invention uses the combination of parenteral and mucosal formulations administered in a prime-boost regimen. Surprisingly, as will be shown in more detail in the examples below, preexisting immunity induced by the prime component does not interfere with the booster component of this novel two-component TB vaccine. Without being bound by theory, it is believed that the basis for the lack of interference may be due to the fact that parenteral vaccines induce relatively poor T-cell responses in the mucosal compartment and only afford partial to negligible protection against mucosal challenges (7-10, 30, 39, 41, 55). Thus, a parenteral vaccine does not induce mucosal T cell responses and does not interfere with the subsequent colonization of the boost in mucosal tissues.

The compositions that are administered contain live, attenuated Mycobacteria. Such “live, attenuated Mycobacteria” include but are not limited to attenuated strains such as BCG, recombinant genetically modified mycobacterial organisms, etc. In some embodiments of the invention, the prime and boost compositions comprise the same live, attenuated Mycobacteria but may be formulated differently, the parenteral composition being formulated in a manner consistent with parenteral administration, and the mucosal composition being formulated in a manner consistent with musocal administration, as described below. However, in other embodiments, a heterologous system is utilized in which some or all of the attenuated Mycobacteria in the parenteral formulation differ from those of the mucosal formulation. In addition, a parenteral formulation (or a mucosal formulation) may include a mixture of more than one type or strain of live, attenuated Mycobacteria. Further, in some cases, the formulations may include entities that encode or otherwise deliver Mycobacterial antigens. Examples include but are not limited to various plasmids, viral vectors (e.g. adenoviral vectors), and non-mycobacterial bacteria that are genetically engineered to encode mycobacterial antigens (e.g. Shigella), etc. Such entities may be included in a formulation instead of live, attenuated Mycobacteria, or in addition to live, attenuated Mycobacteria

Upon administration, the compositions as described herein elicit an immune response against Mycobacterium species, which may be pathogenic. By “elicit an immune response”, we mean that administration of the antigen (one or more types of live, attenuated Mycobacteria) causes the synthesis of antibodies, and/or CD4+ or CD8+ T cell proliferation, and/or cytokine secretion as measured by intracellular cytokine staining, ELISA, or other means well known to those of skill in the art. The compositions may also be used as a vaccine. By “vaccine” we mean that the compositions elicit an immune response which results in protection of the vaccinated host against challenge with a Mycobacterium species (e.g. a pathogenic species) bearing the same or similar antigens as those of the live, attenuated Mycobacteria in the composition. Such protection either wholly or partially prevents or arrests the development of symptoms related to infection, in comparison to non-vaccinated (e.g. adjunct alone) control organisms.

The compositions utilized in the practice of the invention may contain only live, attenuated Mycobacteria, or, alternatively, the compositions may contain a mixture or “cocktail” of different antigenic moieties. For example, the live, attenuated Mycobacteria may be administered in a preparation that also includes other known vaccine components, e.g. components for vaccination against polio, diphtheria, pertussis, etc. In addition, the compositions may be administered in conjunction with other treatment modalities such as substances that boost the immune system, various chemotherapeutic agents, other vaccines, and the like.

The preparation of compositions for both parenteral and mucosal administration is well known to those of skill in the art, and further particulars are discussed below. In general, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The vaccine preparations of the present invention may further comprise an adjuvant, suitable examples of which include but are not limited to Seppic, Quil A, Alhydrogel, etc. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of the live, attenuated Mycobacteria in the formulations may vary. However, in general, the amount in the formulations will be from about 0.01-99%, weight/volume.

In some embodiments of the invention, the parenteral composition is administered first, and the mucosal composition is administered afterwards as the boost. However, this order may be reversed, i.e. the mucosal composition may be administered first, and the parenteral composition may be administered as the boost. Further, in some embodiments, multiple boosts may be administered, and the boosts may be either parenteral or mucosal, or both. Optimization of the time intervals between rounds of administration is discussed below.

Generally, the vaccine regimen of the invention is used to vaccinate mammals such as humans. However, veterinary applications are also contemplated.

### Live Attenuated Mycobacterium Strains

In one embodiment of the invention, each component of the novel two-component TB vaccine is comprised of live attenuated Mycobacterium. The particular live attenuated Mycobacterium strain is not critical to the present invention and can be selected from any of the Mycobacterium species, including but not restricted to M. tuberculosis strain CDC1551 (See, e.g. Griffith et al., Am. J. Respir. Crit. Care Med. August; 152(2):808; 1995), M. tuberculosis strain Beijing (Soolingen et al., 1995), M. tuberculosis strain H37Ra (ATCC#:25177), M. tuberculosis strain H37Rv (ATCC#:25618), M. bovis (ATCC#:19211 and 27291), M. fortuitum (ATCC#:15073), M. smegmatis (ATCC#:12051 and 12549), M. intracellulare (ATCC#:35772 and 13209), M. kansasii (ATCC#:21982 and 35775) M. avium (ATCC#:19421 and 25291), M. gallinarum (ATCC#:19711), M. vaccae (ATCC#:15483 and 23024), M. leprae (ATCC#:), M. marinarum (ATCC#: 11566 and 11567), and M. microtti (ATCC#:11152).

Examples of attenuated Mycobacterium strains include but are not restricted to M. tuberculosis pantothenate auxotroph strain (Sambandamurthy, Nat. Med. 2002 8(10):1171; 2002), M. tuberculosis rpoV mutant strain (Collins et al., Proc Natl Acad Sci USA. 92(17):8036; 1995), M. tuberculosis leucine auxotroph strain (Hondalus et al., Infect. Immun. 68(5):2888; 2000), BCG Danish strain (ATCC #: 35733), BCG Japanese strain (ATCC #: 35737), BCG, Chicago strain (ATCC # 27289), BCG Copenhagen strain (ATCC 4: 27290), BCG Pasteur strain (ATCC #: 35734), BCG Glaxo strain (ATCC #: 35741), BCG Connaught strain (ATCC #: 35745), BCG Montreal (ATCC #: 35746). In addition, the following United States patents, the complete contents of each of which is hereby incorporated by reference, list antigens that may be used in the practice of the invention: U.S. Pat. No. 6,991,797 to Andersen et al.; U.S. Pat. No. 6,596,281 to Gennaro et al., U.S. Pat. No. 6,350,456 to Reed et al.; U.S. Pat. No. 6,290,969 to Reed et al.; U.S. Pat. No. 5,955,356 to Content et al.; and U.S. Pat. No. 5,916,558 to Content et al.

In another preferred embodiment of the present invention, the two-component TB vaccine can include attenuated Mycobacterium strains that carry a passenger nucleotide sequence (“PNS”, i.e. a heterologous or foreign nucleotide sequence originating from another organism). The PNS may encode one or more endosomolytic proteins, such as Listeriolysin (GenBank Accession no. CAA59919 or CAA42639), Escherichia coli Hemolysin (GenBank Accession no. AAC24352 or CAA0535) and Perfringolysin (GenBank Accession no. P19995 or AAA23271), which imparts the ability to degrade the endosome, either partially resulting in leakage of antigens into the cytoplasm, or to the extent that the endosome is ruptured and the Mycobacterium strain escapes this subcellular compartment and resides in the cytoplasm (Hess et al., Proc Natl Acad. Sci., 95:5299-5304; 1998; Grode et al., Clin Invest., 115:2472-2479; 2005).

In a further embodiment of this invention, attenuated Mycobacterium strains are modified to enhance apoptosis, wherein such strains induce strong cellular immune responses. Apoptosis is programmed cell death, which differs dramatically from necrotic cell death in terms of its induction and consequences. In fact, the process by which apoptosis of antigen containing cells results in the induction of potent cellular immunity has been called cross-priming (Heath et al., Immunol Rev 199; 2004; Gallucci et al., Nature Biotechnology. 5:1249; 1999; Albert et al., Nature 392:86; 1998). There are several mechanisms for induction of apoptosis which lead to increased antigen specific cell mediated immunity. Caspase 8-mediated apoptosis leads to antigen specific cellular immune protection (Sheridan et al., Science 277:818; 1997).

Another embodiment of the present invention, therefore, provides attenuated Mycobacterium strains which display enhanced pro-apoptosis properties, such as but not limited to secA1 secreted SodA lacking a leader peptide from Salmonella enteriditis (GenBank Accession no. 1068147), Escherichia coli (GenBank Accession No. 1250070) or Shigella flexneri (GenBank Accession no. 1079977) or alternatively a SodA protein that is naturally non-secreted such as the SodA from Listeria monocytogenes EGD-e (GenBank Accession No. 986791). Such attenuated Mycobacterium strains do not produce extracellular Sod and thus do not suppress host immune responses, yet they do express intracellular Sod, thereby enabling their survival (Edwards et al., Am. J. Respir. Crit. Care Med. 164(12):2213-9; 2001). Alternatively, attenuated Mycobacterium strains which display enhanced pro-apoptosis properties carry an inactivated Rv3238c gene.

Alternatively, expression of Salmonella SopE (GenBank Accession # AAD54239, AAB51429 or AAC02071) or caspase-8 (GenBank Accession # AAD24962 or AAH06737) in the cytoplasm of host cells by attenuated Mycobacterium is a powerful method for inducing programmed cell death in the context of antigens expressed by said attenuated Mycobacterium, invoking high levels of antigen-specific cellular immunity.

Death receptor-5 (DR-5) also known as TRAIL-R2 (TRAIL receptor 2) and TNFR-SF-10B (Tumor Necrosis Factor-Superfamily member 110B) also mediate caspase 8 mediated apoptosis (Sheridan et al., 1997). Reovirus induced apoptosis is mediated by TRAIL-DR5 leading to subsequent clearance of the virus (Clarke et al., J. Virol. 74:8135; 2000). Expression of DR-5, such as human DR-5 (GenBank Accession # BAA33723), herpesvirus-6 (HHV-6) DR-5 homologue (GenBank Accession # CAA58423) etc., by attenuated Mycobacterium in the present invention provides a potent adjuvant effect for induction of antigen-specific cellular immunity against Mtb antigens.

In addition, host antigen presenting cells (such as macrophages and dendritic cells) can also be induced to undergo apoptosis through Fas ligation, which is a strong stimulus for induction of antigen specific cellular immune responses (Chattergoon et al., Nat. Biotechnol. 18:974; 2000). Thus, attenuated Mycobacterium expressing Fas or Fas cytoplasmic domain/CD4 ectodomain fusion protein will induce apoptosis and augment antigen-specific cellular immune responses.

In summary, attenuated Mycobacterium strains which promote the induction of apoptosis provide a powerful tool for the induction of cellular responses that lead to immune mediated cell destruction of Mtb-infected cells, with subsequent elimination, reduction or prevention of the Mtb infection.

In yet another embodiment of the present invention, the two-component TB vaccine can include attenuated Mycobacterium strains that over express at least one Mycobacterium antigen, including but not restricted to Rv0125, Rv0203, Rv0287, Rv0288, Rv0603, Rv1196, Rv1223, Rv1271c, Rv1733c, Rv1738 Rv1804c, Rv1886, Rv2031c, Rv2032, Rv2253, Rv2290, Rv2389c, Rv2626c, Rv2627c, Rv2779c, Rv2873, Rv2875, Rv3017c, Rv3407, Rv3804c, Rv3810, or Rv3841. Alternatively, the over expressed Mycobacterium antigens can be in the form of a fusion protein comprised of one or more said Mycobacterium fusion proteins, such as Mtb72f (14, 60), Hybrid-1 (42, 48), Hyvac-4 (21), etc.

This invention has utility in the development of vaccines against pathogenic Mycobacterium species and in the development of antigen delivery vaccine vectors. A Mycobacterium vector is defined herein as any Mycobacterium strain engineered to express at least one passenger nucleotide sequence (herein referred to as “PNS”) comprised of DNA or RNA and encoding any combination of antigens, immunoregulatory factors or adjuvants, as set forth below. The PNS can be introduced into the chromosome or as part of an expression vector using compositions and methods well known in the art (Jacobs et al., Nature 327:532-535; 1987; Barletta et al., Res Microbiol. 141:931-939; 1990; Kawahara et al., Clin Immunol. 105:326-331; 2002; Lim et al., AIDS Res Hum Retroviruses. 13:1573-1581; 1997; Chujoh et al., Vaccine, 20:797-804; 2001; Matsumoto et al., Vaccine, 14:54-60; 1996; Haeseleer et al., Mol Biochem Parasitol., 57:117-126; 1993).

In the present invention, the Mycobacterium vector may carry a PNS encoding an immunogen, which may be either a foreign immunogen from viral, bacterial and parasitic pathogens, or an endogenous immunogen, such as but not limited to an autoimmune antigen or a tumor antigen. The immunogens may be the full-length native protein, chimeric fusions between the foreign immunogen and an endogenous protein or mimetic, a fragment or fragments of an immunogen that originates from viral, bacterial and parasitic pathogens.

As used herein, “foreign immunogen” means a protein or fragment thereof, which is not normally expressed in the recipient animal cell or tissue, such as, but not limited to, viral proteins, bacterial proteins, parasite proteins, cytokines, chemokines, immunoregulatory agents, or therapeutic agents.

An “endogenous immunogen” means a protein or part thereof that is naturally present in the recipient animal cell or tissue, such as, but not limited to, an endogenous cellular protein, an immunoregulatory agent, or a therapeutic agent. Alternatively or additionally, the immunogen may be encoded by a synthetic gene and may be constructed using conventional recombinant DNA methods known to those of skill in the art.

The foreign immunogen can be any molecule that is expressed by any viral, bacterial, or parasitic pathogen prior to or during entry into, colonization of, or replication in their animal host; the Mycobacterium vector may express immunogens or parts thereof that originate from viral, bacterial and parasitic pathogens. These pathogens can be infectious in humans, domestic animals or wild animal hosts.

The viral pathogens, from which the viral antigens are derived (i.e. the pathogens in which they occur in nature, and from which they originate), include, but are not limited to, Orthomyxoviruses, such as influenza virus (Taxonomy ID: 59771; Retroviruses, such as RSV, HTLV-1 (Taxonomy ID: 39015), and HTLV-II (Taxonomy ID: 11909), Herpes viruses such as EBV Taxonomy ID: 10295); CMV (Taxonomy ID: 10358) or herpes simplex virus (ATCC #: VR-1487); Lentiviruses, such as HIV-1 (Taxonomy ID: 12721) and HIV-2 Taxonomy ID: 11709); Rhabdoviruses, such as rabies; Picornoviruses, such as Poliovirus (Taxonomy ID: 12080); Poxviruses, such as vaccinia (Taxonomy ID: 10245); Rotavirus (Taxonomy ID: 10912); and Parvoviruses, such as adeno-associated virus 1 (Taxonomy ID: 85106).

Examples of viral antigens can be found in the group including but not limited to the human immunodeficiency virus antigens Nef (National Institute of Allergy and Infectious Disease HIV Repository Cat. #183; GenBank Accession # AF238278), Gag, Env (National Institute of Allergy and Infectious Disease HIV Repository Cat. # 2433; GenBank Accession # U39362), Tat (National Institute of Allergy and Infectious Disease HIV Repository Cat. # 827; GenBank Accession # M13137), mutant derivatives of Tat, such as Tat-D31-45 (Agwale et al., Proc. Natl. Acad. Sci. In press. Jul. 8th; 2002), Rev (National Institute of Allergy and Infectious Disease HIV Repository Cat. # 2088; GenBank Accession # L14572), and Pol (National Institute of Allergy and Infectious Disease HIV Repository Cat. # 238; GenBank Accession # AJ237568) and T and B cell epitopes of gp120 (Hanke and McMichael, AIDS Immunol Lett., 66:177; 1999); (Hanke, et al., Vaccine, 17:589; 1999); (Palker et al., J. Immunol., 142:3612?3619; 1989) chimeric derivatives of HIV-1 Env and gp120, such as but not restricted to fusion between gp120 and CD4 (Fouts et al., J. Virol., 74:11427-11436; 2000); truncated or modified derivatives of HIV-1 env, such as but not restricted to gp140 (Stamatos et al., J Virol, 72:9656-9667; 1998) or derivatives of HIV-1 Env and/or gp140 thereof (Binley, et al., J Virol, 76:2606-2616; 2002); (Sanders, et al., J Virol, 74:5091-5100; 2000); (Binley, et al., J Virol, 74:627-643; 2000), the hepatitis B surface antigen (GenBank Accession # AF043578); (Wu et al., Proc. Natl. Acad. Sci., USA, 86:4726?4730; 1989); rotavirus antigens, such as VP4 (GenBank Accession # AJ293721; Mackow et al., Proc. Natl. Acad. Sci., USA, 87:518?522; 1990) and VP7 (GenBank Accession # AY003871; Green et al., J. Virol., 62:1819?1823; 1988), influenza virus antigens such as hemagglutinin or (GenBank Accession # AJ404627; Pertmer and Robinson, Virology, 257:406; 1999); nucleoprotein (GenBank Accession # AJ289872; Lin et al., Proc. Natl. Acad. Sci., 97: 9654-9658; 2000) herpes simplex virus antigens such as thymidine kinase (GenBank Accession # AB047378); (Whitley et al., New Generation Vaccines, 825-854; 2004).

The bacterial pathogens, from which the bacterial antigens are derived, include but are not limited to, Mycobacterium spp., Helicobacter pylori, Salmonella spp., Shigella spp., E. coli, Rickettsia spp., Listeria spp., Legionella pneumoniae, Pseudomonas spp., Vibrio spp., and Borellia burgdorferi.

Examples of protective antigens of bacterial pathogens include the somatic antigens of enterotoxigenic E. coli, such as the CFA/I fimbrial antigen (Yamamoto et al., Infect. Immun., 50:925?928; 1985) and the nontoxic B-subunit of the heat-labile toxin (Klipstein et al., Infect. Immun., 40:888-893; 1983); pertactin of Bordetella pertussis (Roberts et al., Vacc., 10:43-48; 1992), adenylate cyclase-hemolysin of B. pertussis (Guiso et al., Micro. Path., 11:423-431; 1991), fragment C of tetanus toxin of Clostridium tetani (Fairweather et al., Infect. Immun., 58:1323?1326; 1990), OspA of Borellia burgdorferi (Sikand, et al., Pediatrics, 108:123-128; 2001); (Wallich, et al., Infect Immun, 69:2130-2136; 2001), protective paracrystalline-surface-layer proteins of Rickettsia prowazekii and Rickettsia typhi (Carl, et al., Proc Natl Acad Sci USA, 87:8237-8241; 1990), the listeriolysin (also known as “Llo” and “Hly”) and/or the superoxide dismutase (also know as “SOD” and “p60”) of Listeria monocytogenes (Hess, et al., Infect. Immun. 65:1286-92; 1997; (Hess, et al., Proc. Natl. Acad. Sci. 93:1458-1463; 1996); (Bouwer, et al., J. Exp. Med. 175:1467-71; 1992), the urease of Helicobacter pylori (Gomez-Duarte, et al., Vaccine 16, 460-71; 1998); Corthesy-Theulaz, et al., Infection & Immunity 66, 581-6; 1998), and the receptor-binding domain of lethal toxin and/or the protective antigen of Bacillus anthrax (Price, et al., Infect. Immun. 69, 4509-4515; 2001).

The parasitic pathogens, from which the parasitic antigens are derived, include but are not limited to, Plasmodium spp., such as Plasmodium falciparum (ATCC#: 30145); Trypanosome spp., such as Trypanosoma cruzi (ATCC#: 50797); Giardia spp., such as Giardia intestinalis (ATCC#: 30888D); Boophilus spp., Babesia spp., such as Babesia microti (ATCC#: 30221); Entamoeba spp., such as Entamoeba histolytica (ATCC#: 30015); Eimeria spp., such as Eimeria maxima (ATCC# 40357); Leishmania spp. (Taxonomy ID: 38568); Schistosome spp., Brugia spp., Fascida spp., Dirofilaria spp., Wuchereria spp., and Onchocerea spp.

Examples of protective antigens of parasitic pathogens include the circumsporozoite antigens of Plasmodium spp. (Sadoff et al., Science 240:336-337; 1988), such as the circumsporozoite antigen of P. bergerii or the circumsporozoite antigen of P. falciparum; the merozoite surface antigen of Plasmodium spp. (Spetzler et al., Int. J. Pept. Prot. Res., 43:351-358; 1994); the galactose specific lectin of Entamoeba histolytica (Mann et al., Proc. Natl. Acad. Sci., USA, 88:3248-3252; 1991), gp63 of Leishmania spp. (Russell et al., J. Immunol., 140:1274?1278; 1988); (Xu and Liew, Immunol., 84: 173-176; 1995), gp46 of Leishmania major (Handman et al., Vaccine, 18: 3011-3017; 2000), paramyosin of Brugia malayi (Li et al., Mol. Biochem. Parasitol., 49:315-323; 1991), the triose-phosphate isomerase of Schistosoma mansoni (Shoemaker et al., Proc. Natl. Acad. Sci., USA, 89:1842? 1846; 1992); the secreted globin-like protein of Trichostrongylus colubriformis (Frenkel et al., Mol. Biochem. Parasitol., 50:27-36; 1992); the glutathione-S-transferase's of Frasciola hepatica (Hillyer et al., Exp. Parasitol., 75:176-186; 1992), Schistosoma bovis and S. japonicum (Bashir et al., Trop. Geog. Med., 46:255-258; 1994); and KLH of Schistosoma bovis and S. japonicum (Bashir et al., supra, 1994).

As mentioned earlier, the Mycobacterium vector may carry a PNS encoding an endogenous immunogen, which may be any cellular protein, immunoregulatory agent, or therapeutic agent, or parts thereof, that may be expressed in the recipient cell, including but not limited to tumor, transplantation, and autoimmune immunogens, or fragments and derivatives of tumor, transplantation, and autoimmune immunogens thereof. Thus, in the present invention, Mycobacterium vector may carry a PNS encoding tumor, transplant, or autoimmune immunogens, or parts or derivatives thereof. Alternatively, the Mycobacterium vector may carry synthetic PNS's (as described above), which encode tumor-specific, transplant, or autoimmune antigens or parts thereof.

Examples of tumor specific antigens include prostate specific antigen (Gattuso et al., Human Pathol., 26:123-126; 1995), TAG-72 and CEA (Guadagni et al., Int. J. Biol. Markers, 9:53-60; 1994), MAGE-1 and tyrosinase (Coulie et al., J. Immunothera., 14:104-109; 1993). Recently, it has been shown in mice that immunization with non-malignant cells expressing a tumor antigen provides a vaccine effect, and also helps the animal mount an immune response to clear malignant tumor cells displaying the same antigen (Koeppen et al., Anal. N.Y. Acad. Sci., 690:244-255; 1993).

Examples of transplant antigens include the CD3 molecule on T cells (Alegre et al., Digest. Dis. Sci., 40:58-64; 1995). Treatment with an antibody to CD3 receptor has been shown to rapidly clear circulating T cells and reverse cell-mediated transplant rejection (Alegre et al., supra, 1995).

Examples of autoimmune antigens include IAS b chain (Topham et al., Proc. Natl. Acad. Sci., USA, 91:8005-8009; 1994). Vaccination of mice with an 18 amino acid peptide from IAS P chain has been demonstrated to provide protection and treatment to mice with experimental autoimmune encephalomyelitis (Topham et al., supra, 1994).

Mycobacterium Vectors which Express an Adjuvant

It is feasible to construct Mycobacterium vectors that carry PNS encoding an immunogen and an adjuvant, and are useful in eliciting augmented host responses to the vector and PNS-encoded immunogen. Alternatively, it is feasible to construct Mycobacterium “partnered” vectors that carry PNS encoding an adjuvant, which are administered in mixtures with other Mycobacterium vectors that carry PNS encoding at least one immunogen to increase host responses to said immunogen encoded by the other partner Mycobacterium vector.

The particular adjuvant encoded by PNS inserted in said Mycobacterium vector is not critical to the present invention and may be the A subunit of cholera toxin (i.e. CtxA; GenBank Accession no. X00171, AF175708, D30053, D30052,), or parts and/or mutant derivatives thereof (E.g. the A1 domain of the A subunit of Ctx (i.e. CtxA1; GenBank Accession no. K02679)), from any classical Vibrio cholerae (E.g. V. cholerae strain 395, ATCC # 39541) or El Tor V. cholerae (E.g. V. cholerae strain 2125, ATCC # 39050) strain. Alternatively, any bacterial toxin that is a member of the family of bacterial adenosine diphosphate-ribosylating exotoxins (Krueger and Barbier, Clin. Microbiol. Rev., 8:34; 1995), may be used in place of CtxA, for example the A subunit of heat-labile toxin (referred to herein as EltA) of enterotoxigenic Escherichia coli (GenBank Accession # M35581), pertussis toxin SI subunit (e.g. ptxS1, GenBank Accession # AJ007364, AJ007363, AJ006159, AJ006157, etc.); as a further alternative the adjuvant may be one of the adenylate cyclase-hemolysis of Bordetella pertussis (ATCC #8467), Bordetella bronchiseptica (ATCC #7773) or Bordetella parapertussis (ATCC #15237), e.g. the cyaA genes of B. pertussis (GenBank Accession no. X14199), B. parapertussis (GenBank Accession no. AJ249835) or B. bronchiseptica (GenBank Accession no. Z37112).

Mycobacterium Vectors which Express an Immunoregulatory Agent

Yet another approach entails the use of Mycobacterium vectors that carry at least one PNS encoding an immunogen and a cytokine, which are used to elicit augmented host responses to the PNS-encoded immunogen Mycobacterium vector. Alternatively, it is possible to construct a Mycobacterium vector that carries a PNS encoding said cytokine alone, which are used in admixtures with at least one other Mycobacterium vector carrying a PNS encoding an immunogen to increase host responses to PNS-encoded immunogens expressed by the partner Mycobacterium vector.

The particular cytokine encoded by the Mycobacterium vector is not critical to the present invention includes, but not limited to, interleukin-4 (herein referred to as “IL-4”; GenBank Accession no. AF352783 (Murine IL-4) or NM_{—}000589 (Human IL-4)), IL-5 (GenBank Accession no. NM_{—}010558 (Murine IL-5) or NM_{—}000879 (Human IL-5)), IL-6 (GenBank Accession no. M20572 (Murine IL-6) or M29150 (Human IL-6)), IL-10 (GenBank Accession no. NM_{—}010548 (Murine IL-10) or AF418271 (Human IL-10)), 11-12p40 (GenBank Accession no. NM_{—}008352 (Murine IL-12 p40) or AY008847 (Human IL-12 p40)), IL-12p70 (GenBank Accession no. NM_{—}008351/NM_{—}008352 (Murine IL-12 p35/40) or AF093065/AY008847 (Human IL -12 p35/40)), TGFb (GenBank Accession no. NM_{—}011577 (Murine TGFb1) or M60316 (Human TGFb1)), and TNFa GenBank Accession no. X02611 (Murine TNFa) or M26331 (Human TNFa)).

### Construction and Propagation of Mycobacterium Strains

The above-described Mycobacterium strains can be made using standard molecular biology techniques well known to the art. For example, restriction endonucleases (herein “REs”); New England Biolabs Beverly, Mass.), T4 DNA ligase (New England Biolabs, Beverly, Mass.) and Taq polymerase (Life Technologies, Gaithersburg, Md.) are used according to the manufacturers' protocols; Plasmid DNA is prepared using small-scale (Qiagen Miniprep® kit, Santa Clarita, Calif.) or large-scale (Qiagen Maxiprep® kit, Santa Clarita, Calif.) plasmids DNA purification kits according to the manufacturer's protocols (Qiagen, Santa Clarita, Calif.); Nuclease-free, molecular biology grade milli-Q water, Tris-HCl (pH 7.5), EDTA pH 8.0, 1M MgCl₂, 100% (v/v) ethanol, ultra-pure agarose, and agarose gel electrophoresis buffer are purchased from Life Technologies, Gaithersburg, Md. RE digestions, PCRs, DNA ligation reactions and agarose gel electrophoresis is conducted according to well-known procedures (Sambrook, et al., Molecular Cloning: A Laboratory Manual. 1, 2, 3; 1989); (Straus, et al., Proc Natl Acad Sci USA. March; 87(5):1889-93; 1990). Nucleotide sequencing to verify the DNA sequence of each recombinant plasmid described in the following sections was accomplished by conventional automated DNA sequencing techniques using an Applied Biosystems automated sequencer, model 373A.

PCR primers may be purchased from commercial vendors such as Sigma (St. Louis, Mo.) and are synthesized using an Applied Biosystems DNA synthesizer (model 373A). PCR primers are used at a concentration of 150-250 mM and annealing temperatures for the PCR reactions are determined using Clone manager software version 4.1 (Scientific and Educational Software Inc., Durham, N.C.). PCRs are conducted in a Strategene Robocycler, model 400880 (Strategene, La Jolla, Calif.). The PCR primers for the amplifications are designed using Clone Manager® software version 4.1 (Scientific and Educational Software Inc., Durham, N.C.). This software enabled the design PCR primers and identifies RE sites that are compatible with the specific DNA fragments being manipulated. PCRs are conducted in a thermocycler device, such as the Strategene Robocycler, model 400880 (Strategene), and primer annealing, elongation and denaturation times in the PCRs are set according to standard procedures (Straus et al., supra, 1990). The RE digestions and the PCRs are subsequently analyzed by agarose gel electrophoresis using standard procedures (Straus et al., supra 1990); (Sambrook, et al., supra, 1989). A positive clone is defined as one that displays the appropriate RE pattern and/or PCR pattern. Plasmids identified through this procedure can be further evaluated using standard DNA sequencing procedures, as described above.

Escherichia coli strains, such as DH5a and Top10, may be purchased from Invitrogen (Gaithersburg, Md.) and serve as initial host of the recombinant plasmids described in the examples below. Recombinant plasmids are introduced into E. coli strains by electroporation using an high-voltage eletropulse device, such as the Gene Pulser (BioRad Laboratories, Hercules, Calif.), set at 100-200W, 15-25 mF and 1.0-2.5 kV, as described (Ausubel et al, supra). Optimal electroporation conditions are identified by determining settings that result in maximum transformation rates per mg DNA per bacterium.

Laboratory bacterial strains are grown on tryptic soy agar (Difco, Detroit, Mich.) or in tryptic soy broth (Difco, Detroit, Mich.), which are made according to the manufacturer's directions. Unless stated otherwise, all bacteria are grown at 37° C. in 5% CO₂ with gentle agitation. When appropriate, the media are supplemented with antibiotics (Sigma, St. Louis, Mo.). Bacterial strains are stored at −80° C. suspended in (Difco) containing 30% glycerol (v/v; Sigma, St. Louis, Mo.) at ca. 10⁹ colony-forming units (herein referred to as “cfu”) per ml.

The prior art also teaches methods for introducing altered alleles into Mycobacterium strains and those skilled in the art will be capable of interpreting and executing said methods (Parish et al., Microbiology, 146:1969-1975; 2000). A novel method to generate an allelic exchange plasmid entails the use of synthetic DNA. The advantage of this approach is that the plasmid product will have a highly defined history and will be 21 CFR compliant (21 CFR207.31, 607), whereas previously used methods, although effective, have poorly documented laboratory culture records and thus are unlikely to be 21 CFR compliant. Compliance with said regulation is essential if a product is to be licensed for use in humans by United States and European regulatory authorities (21CFR 601.2, 600-680).

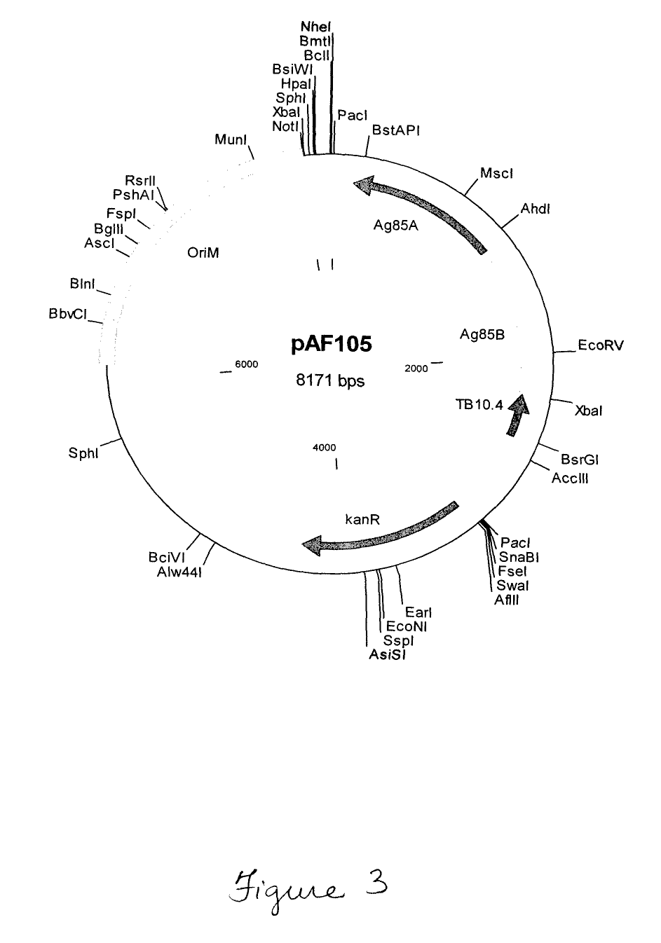

A suicide vector for allelic exchange in Mycobacterium is a plasmid that has the ability to replicate in E. coli strains but is incapable of replication in Mycobacterium spp., such as Mtb and BCG. The specific suicide vector for use in allelic exchange procedures in the current invention is not important and can be selected from those available from academic (Parish et al., supra, 2000) and commercial sources. A preferred design of a suicide plasmid for allelic exchange is shown in FIG. 2. The plasmid is comprised of following DNA segments: An oriE sequence for the plasmid to replicate in E. coli (GenBank Accession #: L09137), a Kanamycin-resistant sequence for selection in both E. coli and Mycobacterium (GenBank Accession #: AAM97345), and an additional antibiotic selection marker (e.g. the zeocin-resistance gene (GenBank Accession #: AAU06610)), which is under the control of a Mycobacterium promoter (e.g. the hsp60 promoter). The second antibiotic selection marker is not essential but is included to enable double selection to prevent outgrowth of spontaneous kanamycin-resistant isolates during the allelic exchange process (Garbe et al., Microbiology. 140:133-138; 1994).

Construction of such a suicide vectors can be accomplished using standard recombinant DNA techniques as described herein. However, current regulatory standards (e.g. 21 CFR) have raised the specter of introducing prion particles acquired from materials exposed to bovine products containing transmissible spongiform encephalitis (BSE) prion particles. To avoid introducing materials (e.g. DNA sequences) into the target strain of unknown origin, therefore, it is preferable that all DNA in the suicide vector are made synthetically by commercial sources (e.g. Picoscript, Inc.). Accordingly, a preferred method for constructing suicide vectors is to assemble a plan of the DNA sequences using DNA software (e.g. Clone Manager), then to synthesize the DNA on a fee-for-service basis by any commercial supplier that offer such a service (e.g. Picoscript Inc.). The suicide vector carries sequences encoding at least one antibiotic selection marker for positive selection of merodiploids. For negative selection during the excision stage of allelic exchange, a sacB gene (GenBank Accession # AAA22724 or AAA72302), which imparts a sucrose-sensitive phenotype, is included to enrich cultures with strains that have undergone the final DNA recombination step and completed the allelic exchange.

### Cultivation of Mycobacterium Strains

Selected Mycobacterium strains are cultured in liquid media, such as Middlebrook 7H9 or Saulton Synthetic Medium, preferably at 37° C. The strains can be maintained as static or agitated cultures. In addition the growth rate of BCG can be enhanced by the addition of oleic acid (0.06% v/v; Research Diagnostics Cat. No. 01257) and detergents such as Tyloxapol (0.05% v/v; Research Diagnostics Cat. No. 70400). The purity of Mycobacterium cultures can be evaluated by evenly spreading 100 mcl aliquots of the Mycobacterium culture serially diluted (e.g. 10-fold steps from Neat—10-8) in phosphate buffered saline (herein referred to PBS) onto 3.5 inch plates containing 25-30 ml of solid media, such as Middlebrook 7H10. In addition, the purity of the culture can be further assessed using commercially available medium such as Thioglycolate medium (www.sciencelab.com, Cat 41891) and Soybean-Casin medium (BD, Cat #: 211768) as described in 21CFR610.12

Mycobacterium seed lots are stored at −80° C. at a density of 0.1−2×10⁷ cfu/ml. The liquid cultures are typically harvested at an optical density (at 600 nm) of 0.2-4.0 relative to a sterile control; the cultures are placed into centrifuge tubes of an appropriate size and the organisms are subjected to centrifugation at 8,000×g for 5-10 min. The supernatant is discarded and the organisms are resuspended in storage solution comprised of Middlebrook 7H9 containing 10-30% glycerol at a density of 0.1−2×10⁷ cfu/ml. These suspensions are dispensed into sterile 1.5 ml boron silicate freezer vials in 1 ml aliquots and then placed at −80° C.

### Manufacturing of the Two-Component TB Vaccine

i) Premaster Seed characterization

Prior to manufacturing the Master Seed Bank (which is defined as a collection of cells of uniform composition derived from a single tissue or cell which is cryopreserved in aliquots stored in the liquid or vapor phase of liquid nitrogen), the purity of Mycobacterium vaccine cultures is reevaluated by evenly spreading 100 ml aliquots of the cultures serially diluted (e.g. 10-fold steps from Neat—10⁻⁸) in phosphate buffered saline (PBS) onto 8.75 cm plates containing 25-30 ml of solid media (Middlebrook 7H10). The purity of the cultures is also assessed using commercially available kits. PCR, restriction endonuclease analysis of plasmid DNA and DNA hybridization are used to confirm that the desired genotype is present in each Mycobacterium isolate. All PCR-generated DNA fragments will be sequenced by automated dideoxynucleotide sequencing techniques to confirm the presence of full-length genes.

The ability of candidate Mycobacterium strains to over express TB antigens or express foreign antigens will be examined as follows. The strain will be cultured as described above. When the culture reaches mid-log phase—stationary phase, whole-cell lysates and culture supernatants filtered through 0.2-mm membrane filters, will be prepared as previously described (31). The whole-cell lysates and culture filtrate proteins (CFPs) will be separated on 10-15% SDS-PAGE gels, transferred to nylon filters, stained with PfoA-specific rabbit serum (diluted 1000- to 5000-fold in PBS) and visualized using chemiluminescent immunodetection techniques. Expression of the antigens will be assessed by separating the whole-cell lysates and CFPs on 10-15% SDS-PAGE gels, transferred to nylon filters, stained with mAbs specific for the protein of interest and visualized using chemiluminescent immunodetection techniques.

To assess the secretion of endosomolytic proteins, such as Llo and PfoA, by candidate Mycobacterium vaccine strains, colonies are selected and grown to mid-logarithmic phase, as described above. The whole-cell lysates will be prepared as described (Anacker et al., J. Immunol., 98:1265-73; 1967; Calaco et al., Biochem Soc Trans., 32:626-8; 2004) and culture supernatants of these cultures will be collected and filtered through 0.2-mm membrane filters, as previously described (31). The whole-cell lysates and culture filtrate proteins will be separated on 10-15% SDS-PAGE gels, transferred to nylon filters, stained with PfoA-specific rabbit serum (diluted 1000- to 5000-fold in PBS) and visualized using chemiluminescent immunodetection techniques. The PfoA protein is ˜56 kDa and will be detectable in supernatants derived from cultures of rBCG-Pfo+ strains. In addition, the hemolytic activity of serial dilutions of the rBCG-Pfo+ supernatants and whole bacterial suspensions in PBS containing 0.1% BSA will be confirmed using sheep erythrocytes as described previously (27). A positive result in this assay correlates with the endosome-escape phenotype (27, 52).

### ii) Preparation of a Master Seed

The Master Seed will be produced in a class C clean room. All of the equipment that will be used to produce Master Seed will be validated. The opening and closing of the vials, flask etc will be performed in a Biosafety cabinet (class 100). A validated steam sterilizer (autoclave) will be used to sterilize the medium, flasks and the fermentor component. Aliquots of the premaster seed will be used to inoculate five 2-liter flasks containing 500 ml Modified Middlebrook medium each. The cultures will be incubated at 37° C. in a gyratory shaker set to oscillate at 120 rpm. After completion of the growth the Master Seed glycerol will be added to a final concentration of 10% (v/v) and 1 ml aliquots will be stored in cryovials at −80° C.

iii) Master Seed Characterization

The assays that will be used to characterize and QC the Master Seed are shown in the table below.

TABLE 1
QC and release of rBCG-HIV Master Seed

| Test/Study | Test method | Type |
|---|---|---|
| Antibiotic Sensitivity | Clinical Screen | Release |
| Buffer composition, | BioAnalyzer | Release |
| osmolarity | | |
| Characterization, colony | Plate culture, solid medium | Release |
| morphology | | |
| Characterization, genetic | Microarray and proteome analysis | QC |
| and phenotype | | |
| HIV antigen expression | Quantitative PAGE or Western Blot | Release |
| HIV gene stability | PCR | QC |
| Expression, pfo | SRBC hemolytic Assay | Release |
| Potency, CFU | Plate dilution | QC |
| Stability/Potency | Plate culture, solid medium | QC |
| Sterility/Bioburden | Direct Inoculation | Release |

iv) cGMP Production of Clinical Trial Material

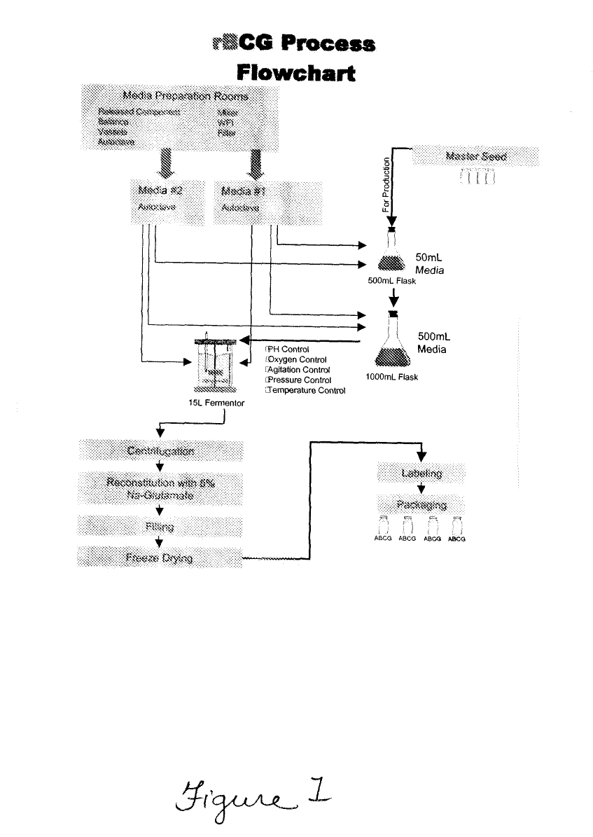

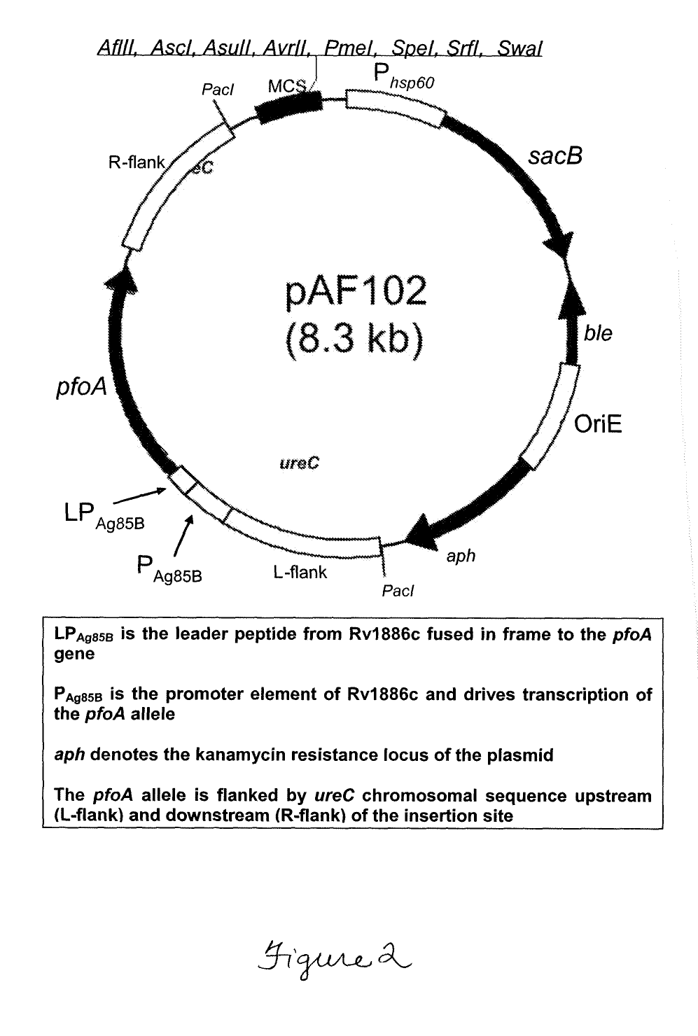

An outline of the two-component TB vaccine manufacturing process is shown in FIG. 1. The facility is designed to meet all regulatory requirements for phase 1 through to product launch. A gowning room and airlock provide a barrier for personnel entering the facility, and transferring equipment in and out of the classified area. Biosafety cabinet (class 100) in manufacturing facility is used for aseptic transfer of inocula within the manufacturing facility class 100,000 room. The opening and closing of the vials, flask etc are performed in a biosafety cabinets (class 100). A validated steam sterilizer (autoclave) is used to sterilize the medium, flasks and the fermentor component. The facility and environmental monitoring system are validated.

Prior to manufacturing the phase 1 clinical trial material, the environmental monitoring, and sanitation Standard Operating Procedures (SOPs) are validated. In addition, the aseptic process is validated by conducting triplicate test runs using trypticase soy broth as the transfer fluid in distinct stages of the simulated sterile manufacturing operation for the vaccine production process.

The inocula are prepared in class C room and the inoculation of cultures during the inoculum preparation is conducted within a class 100 Biosafety cabinet. To prepare the inoculum, the bacterial Master Seed is expanded from 1 ml to 50 ml, then to 500 ml in a shaker incubator at 37° C. The 500 ml culture then is used to inoculate a 20 L fermentor. Fermentation is performed in 10 L of medium (See above), which is sterilized in a validated autoclave for 1 hr at 121° C. prior to inoculation. The temperature is controlled during fermentation at 37° C. and mixing is achieved with two six bladed, flat blade impellers operating at 100-300 rpm and an appropriate aeration rate to maintain 20% dissolved oxygen rate in the bacterial culture in the fermentor. The pH of the culture broth in fermentor is monitored using a sterile pH electrode attached to a pH controller with a set-point limit of pH 6.8-7.2. The pH is controlled automatically by adding HCl or NaOH by the on-of PID activation of peristaltic pump. To monitor the biomass, samples are taken on a daily basis throughout the fermentation run and the biomass is determined by measuring optical density at 540 nm. The levels of glycerol, glucose and other components in the cell-free bacterial culture medium are determined by Biolyzer.

### v) Harvesting

After completion of the growth in fermentor, the culture is collected aseptically in sterilized centrifuge tube and centrifuge to collect the biomass. The biomass will be resuspended in a washing buffer and harvested by centrifugation. A portion of the washed bacteria is resuspended to a concentration of 5×10⁵ cfu/ml in the formulation solution. The remainder is stored as bulk material in medium containing 10% (v/v) glycerol.

### vi) Sterile-Fill and Lyophilization of the Product

The two-component TB vaccine is formulated (See details below) and QC tested, then sterile filled and lyophilized. One ml aliquots containing a single human dose of vaccine suspended in formulation solution are transferred manually into 2 mL amber type I glass vials using validated process and quality controlled for fill volume. Lyophilization is done as a single run as described (Hubeau et al., Clin. Exp. Allergy, 33:386-93; 2003; Kawahara et al., Clin. Immunol., 105:326-31; 2002; Gheorghiu et al., Dev Biol Stand., 87:251-261; 1996). The closure is a slotted chlorobutyl rubber stopper secured with a 20 mm center tear-off aluminum seal. Each vial contains an extractable single-dose of the product.

vii) Quality Control and Release of Candidate Vaccines

The basic test requirements for live Mycobacterium vaccines are specified by the U.S. FDA, European countries (EMEA) and are further guided internationally by guidelines from the World Health Organization. It is expected that a two-component TB vaccine will have to meet all the testing currently required for BCG vaccines. It is also expected that two-component TB vaccines will have to meet functional tests specific for the antigens expressed by the vaccine. In addition, the two-component TB vaccine will have to meet investigational safety testing currently required by both the U.S. FDA and the EMEA.

The proposed testing plan during the manufacture of the two-component TB vaccine is designed to meet current good manufacturing practices for 1) quality control, 2) regulatory testing requirements for BCG vaccines, 3) additional testing for expressed enzyme/antigens, and meet all 4) investigational drug safety testing requirements for phase I clinical trials.

TABLE 2
Release testing of frozen bulk product

| Test/Study | Test method | Type |
|---|---|---|
| Buffer composition, osmolarity, pH | BioAnalyzer | Release |
| Glycerol Content | BioAnalyzer | Release |
| Identity | Dilution Plating | Release |
| Potency, CFU | Dilution Plating | Release |

TABLE 3
Release testing of finished product

| Test/Study | Test method | Type |
|---|---|---|
| Endotoxin | Chromogenic LAL assay | Release |
| Sterility | Direct inoculation | Release |
| Buffer composition, | BioAnalyzer | Release |
| osmolarity | | |
| Characterization, colony | Plate culture, solid medium | Release |
| morphology | | |
| Characterization, genetic | Microarray and proteome | Release |
| and phenotype | analysis | |
| Expression, antigen | Quantitative PAGE or Western | Release |
| | Blot | |
| Residual Cytolysin, (pfo) | SRBC hemolytic Assay | Release |
| Potency, CFU | Plate dilution | QC |
| Stability/Potency | Plate culture, solid medium | QC |
| Fill volume (Target volume | Weight | Release |
| 0.5 mL) | | |
| Residual Moisture(lyo prep) | Karl-Fischer | QC |
| Appearance | Visual inspection | Release |
| Label check | Visual inspection | Release |
| Guinea Pig Skin Test | FDA Requirement | Release |

TABLE 4
Release testing of clinical trial material

| Test/Study | Test method | Specification | Type |
|---|---|---|---|
| Modified General | FDA | As specified | Safety |
| Safety | | | |
| Guinea Pig Freedom | FDA Requirement | As specified | Release |
| from Virulent Mtb | | | |
| Acute Toxicity | EU | As specified | Safety |
| Acute & Extended | FDA | As specified | Safety |
| Toxicity | | | |

### Formulation Strategies

i) Parenteral components

“Parenteral component”, as used herein, refers to a formulation that is suitable for administration for example, subcutaneously, intradermally or intramuscularly. Such an administration may be carried out by any means known to those of skill in the art, for example by injection with a needle, by air gun, rotary lancet, “Mono-vacc” style devices, or any other suitable device, etc. The strategies for vaccine formulation are structured on studies to determine maximum viability and stability throughout the manufacturing process. This includes determination maximum organism viability (live to dead) during culture utilizing a variety of commonly used medium for the culture of Mycobacterium to include the addition of glycerol, sugars, amino acids, and detergents or salts. After culture cells are harvested by centrifugation or tangential flow filtration and resuspended in a stabilizing medium that allows for protection of cells during freezing, freeze-drying or foam drying processes. Commonly used stabilizing agents include sodium glutamate, or amino acid or amino acid derivatives, glycerol, sugars or commonly used salts. The final formulation will provide sufficient viable organism to be delivered by intradermal, subcutaneous or intramuscular injection, with sufficient stability to maintain an adequate shelf-life for distribution and use.

ii) Mucosal components

“Mucosal component”, as used herein, is a formulation that is suitable for administration orally (e.g. by mouth by ingesting a liquid or solid form, or by ingestion of a food product containing the antigenic component), nasally (e.g. by inhalation or drops), rectally (e.g. by suppositories or liquid), etc. Formulation of the mucosal component is dependent on the target mucosal route of administration. The mucosal components are generally administered along with a pharmaceutically acceptable carrier or diluent. The particular pharmaceutically acceptable carrier or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al., J. Clin. Invest., 79:888-902; 1987); (Black et al., J. Infect. Dis., 155:1260-1265; 1987), or bicarbonate buffer (pH 7.0) containing ascorbic acid, lactose, and optionally aspartame (Levine et al., Lancet, II:467?470; 1988). Examples of carriers include proteins, e.g., as found in skim milk, sugars, e.g., sucrose, or polyvinylpyrrolidone. Typically these carriers would be used at a concentration of about 0.1-90% (w/v) but preferably at a range of 1-10% (w/v). In addition, oral formulations can include commercially available products, such as CeraVacx (Cera Inc, Baltimore Md.), which are known to improve the survival of live oral bacterial vaccines following oral administration (Cohen et al., Infect. Immun., 70:1965-1970; 2002; Sack et al., Infect Immun., 65:2107-2111; 1997). In addition oral formulations can be delivered in enteric coated capsule for passage through the stomach.

### Preclinical Evaluation of TB Vaccine Components

### i) General Safety Test

BALB/c mice in groups of six are infected intraperitoneally with 2×10⁶ CFU of the Mycobacterium strain(s) of interest and the analogous parental strains. The animals are monitored for general health and body weight for 14 days post infection. Animals that receive the Mycobacterium strains remain healthy, and neither lose weight nor display overt signs of disease during the observation period.

### ii) Virulence Of Novel Mycobacterium Strains in Immunocompetent Mice

Groups of 15 immunocompetent BALB/c mice are inoculated intravenously with 2×10⁶ cfu of the Mycobacterium strain. At day one post infection, three mice in each group will be sacrificed and CFU in spleen, lung and live are analyzed to ensure each animal has equal infection dose. At week 4, 8, 12, and 16 post infection, three mice in each group are sacrificed and CFU in spleen, live and lung are obtained to assess the in vivo growth of the Mycobacterium strains as compared to the parental Mycobacterium strain. Mycobacterium strains are expected to display reduced virulence to that of wild-type Mycobacterium.

iii) Stringent Safety Test In Immunocompromised Mice

Immunocompromised mice possessing the SCID (severe combined immunodeficiency) in groups of 10 are infected intravenously with 2×10⁶ cfu of the 5 Mycobacterium strain and the wild-type Mycobacterium strain, respectively. One day after infection, three mice in each group are sacrificed and cfu in spleen, liver and lung is assessed to verify the inoculation doses. The remaining seven mice in each group are monitored for general health and body weight. The survival of these mice is followed and attenuation is verified if the survival of Mycobacterium-infected mice is prolonged over the survival of mice inoculated with the wild-type strain.

### iv) Guinea Pig Safety Test

The safety of attenuated Mycobacterium strains is also assessed in the guinea pig model in comparison to BCG (E.g. BCG Copenhagen), which has a well-established safety profile in humans. First, the effect of the vaccine on the general health status of the animals is examined, including weight gain. Guinea pigs are immunized intramuscularly with 10⁷ (100× of vaccination dose) cfu of the recombinant and parental strains, and the animals are monitored for general health and body weight for six weeks. Post mortem examination is performed for animals that die before the six weeks period. All animals are sacrificed at the end of six weeks post infection and gross pathology is performed. There is no body weight loss, no abnormal behavior and all organs appear normal at the six week necropsy. A Mycobacterium strain is deemed attenuated when no adverse health effects are observed in animals inoculated with said attenuated Mycobacterium strain, and animals gain weight at the normal rate compared to animals inoculated with a reference BCG strain.

At the same time, the number of viable bacteria in animal organs are monitored. Guinea pigs are immunized with either BCG or attenuated Mycobacterium strain. At 2, 4, 6, 8 and 10 weeks after inoculation, groups of 5 animals are euthanized and tissues including the regional (inguinal) lymph nodes, lungs, spleen and liver are harvested, homogenized and the numbers of viable BCG or attenuated Mycobacterium are determined by plate count as described (Turner et al., Infect. Immun., 68:3674-3679; 2000; McMurray et al., Infect. Immun. 50:555-559; 1985; Wiegeshaus et al., Am. Rev. Respir. Dis., 102:422-429; 1970).

### v) Toxicity Test

To evaluate the toxicity of the attenuated Mycobacterium strains, groups of 12 guinea pigs are vaccinated intradermally with one dose four times higher, one dose equivalent to and one dose four times lower than a single human dose of the attenuated Mycobacterium strains, BCG or saline respectively. Three days post vaccination 6 animals in each group are sacrificed to access the acute effects of the vaccine on these animals. At day 28 post vaccination, the remaining six animals in each group are sacrificed to evaluate the chronic effects of attenuated Mycobacterium on the animals. At both time points, the body weight of each animal is obtained; Gross pathology and appearance of the injection sites are examined. Blood is taken for blood chemistry, and the histopathology of the internal organs and injection sites are performed. Attenuated Mycobacterium strains are deemed safe if the toxicity of said strains is equivalent to or less than the toxicity of BCG.

### Characterization of Vaccine Potency

### i) Induction of Cutaneous Delayed-Type Hypersensitivity (DTH).

Specific pathogen free (SPF) guinea pigs will be immunized intradermally with 103 attenuated Mycobacterium or BCG. Nine weeks after immunization, the animals will be shaved over the back and inject intradermally with 10 μg of PPD in 100 μl of phosphate buffered saline. After 24 hr, the diameter of hard induration is measured. Attenuated Mycobacterium strains are expected to induce the DTH equal or greater than that induced by the reference BCG strain.

### ii) Murine Protection Study

To determine the potency of the novel two-component TB vaccine against an Mtb challenge, groups of 13 C57B1/6 mice (female, 5-6 weeks of age) are immunized in with priming component of the two-component TB vaccine, BCG or saline. Typically, 10⁶ cfu of the priming component and BCG control are administered intradermally. However, the priming component can also be administered by a mucosal route of inoculation, preferably the oral at a dose of 10⁴-10⁹ cfu, preferably 10⁶-10⁷ cfu. The formulation of the oral priming component is described above and elsewhere (Adwell et al., Vaccine, 22:70-76; 2003; Buddle et al., Vaccine, 23:3581-3589; 2005).

Six to twenty-four weeks, preferably 10 to 17 weeks, most preferably 17 weeks after the prime the mice vaccinated with the priming component of the two-component TB vaccine are boosted with the boosting component of the two component TB vaccine. Animals that received a parenteral prime are boosted by the mucosal route, preferably the oral route; whereas animals that received a mucosal prime are boosted by the parenteral route. The parenteral boosting component is administered subcutaneously, intradermally or intramuscularly, preferably intradermally at a dose of 10⁶ cfu. The mucosal boosting component is administered by a mucosal route of inoculation, preferably the oral route at a dose of 10⁴-10⁹ cfu, preferably 10⁶-10⁷ cfu.

Ten weeks after the final vaccination, mice are challenged with Mtb Erdman strain (or H37Rv Kan-resistant strain) by an aerosol generated from a 10-ml single-cell suspension containing a total of 10⁷ cfu of the challenge strain, a dose that delivers 100 live bacteria to the lungs of each animal, as described previously (Turner et al., Infect. Immun., 68:3674-3679; 2000; McMurray et al., Infect. Immun. 50:555-559; 1985; Wiegeshaus et al., Am. Rev. Respir. Dis., 102:422-429; 1970). The experimental animals are monitored for survival along with unchallenged animals. Following the challenge, the animals are also monitored for weight loss and general health. At day one after challenge, three mice in each group are sacrificed for lung cfu to confirm challenge dose and one is sacrificed for spleen and lung histopathology. Then five weeks after challenge, nine animals in each group are sacrificed, and histopathology and microbiology analysis of the animal are performed. Lung and spleen tissues from six mice are evaluated for cfu counts (plates with selection supplements are used to distinguish the vaccine strain from the challenge strain). If challenged with H37Rv-kan resistant strain, Kan or TCH are used to distinguish the challenge strain from the vaccine strain. If Mtb Erdman strain is used to challenge, TCH is used to distinguish vaccine strain from the challenge strain (BCG is susceptible, but Mtb is naturally resistant).

iii) Guinea Pig Challenge Study

To further characterize the potency of the attenuated Mycobacterium vaccines against Mtb challenge, guinea pigs (young adult SPF Hartley, 250-300 grams, male) are immunized in groups of 12, with priming component of the two-component TB vaccine, BCG or saline. Typically, 10⁶ cfu of the priming component and BCG control are administered intradermally. However, the priming component can also be administered by a mucosal route of inoculation, preferably the oral at a dose of 10⁴-10⁹ cfu, preferably 10⁶-10⁷ cfu. The formulation of the oral priming component is described above and elsewhere (Adwell et al., Vaccine, 22:70-76; 2003; Buddle et al., Vaccine, 23:3581-3589; 2005).

Six to twenty-four weeks, preferably 10 to 17 weeks, most preferably 17 weeks after the prime the guinea pigs vaccinated with the priming component of the two-component TB vaccine are boosted with the boosting component of the two component TB vaccine. Animals that received a parenteral prime are boosted by the mucosal route, preferably the oral route; whereas animals that received a mucosal prime are boosted by the parenteral route. The parenteral boosting component is administered subcutaneously, intradermally or intramuscularly, preferably intradermally at a dose of 10⁶ cfu. The mucosal boosting component is administered by a mucosal route of inoculation, preferably the oral at a dose of 10⁴-10⁹ cfu, preferably 10⁶-10⁷ cfu.

At 10 weeks after the final immunization, immunized animals are challenged by aerosol with the Mtb by an aerosol generated from a 10-ml single-cell suspension containing a total of 10⁷ cfu of Mtb; this procedure delivers ˜100 live bacteria to the lungs of each animal, as described previously (Brodin et al., 2004). Following challenge, the animals are monitored for survival along with a healthy group of unvaccinated, unchallenged animals. Following the challenge, the animals are monitored for weight loss and general health. Six animals in each group are sacrificed at 10 weeks post challenge and remaining six in each group at 70 weeks post challenge for long term evaluation. At both time points, histopathology and microbiology analysis of the animal is performed. Lung and spleen tissues are evaluated for histopathology and cfu count (plates with selection supplements are used to distinguish vaccine strain from challenge strain). If challenge with H37Rv-kan resistant strain, Kan or TCH are used to distinguish challenge strain from the vaccine strain; if Mtb strain Erdman is used as a challenge, TCH (BCG is susceptible but Mtb is naturally resistant) are used to distinguish vaccine strain from the challenge strain. Sham immunized animals are expected to die most rapidly after challenge. In contrast, animals immunized with the novel two-component TB vaccine survive longer than the BCG-immunized animals.

### iv) Primate Safety and Challenge Study

The Rhesus macaque serves as a useful model for evaluation of vaccines against Mtb. The genetic similarities between humans and non-human primates, and the similar clinical and pathologic manifestations of TB in these species has made this model attractive for experimental studies of TB disease and vaccine efficacy.

This model, characterized by the development of lung cavitation, appears to be applicable to human TB. The course of infection and disease is followed by X-ray and weight loss, as well as a variety of hematological tests, including erythrocyte sedimentation rate (ESR), peripheral blood mononuclear cell (PBMC) proliferation and cytokine production, cytotoxic T lymphocytes (CTL) activity, and antibody responses. Following infection with Mtb the monkey develops lung pathology with characteristic lesions, and, depending on the challenge doses, death from acute respiratory infection occurs within four-to six months after infection.

The aim of this study is to evaluate the potency of a BCG standard vaccine vs the two-component TB vaccine of the present invention. The study comprises three groups of 10 animals designed as follows: one group each comprising BCG, a two-component TB vaccine and saline.

Inocula of 10⁶ cfu of the priming component of the two-component TB vaccine and BCG control are administered parenterally, either intramuscularly, subcutaneously or intradermally, preferably intradermally. However, the priming component can also be administered by a mucosal route of inoculation, preferably the oral at a dose of 10⁴-10⁹ Cfu, preferably 10⁶-10⁷ cfu. The formulation of the oral priming component is described above and elsewhere (Adwell et al., Vaccine, 22:70-76; 2003; Buddle et al., Vaccine, 23:3581-3589; 2005).

Six to twenty-four weeks, preferably 10 to 17 weeks, most preferably 17 weeks after the prime the Rhesus macaques vaccinated with the priming component of the two-component TB vaccine are boosted with the boosting component of the two component TB vaccine. Animals that received a parenteral prime are boost by the mucosal route, preferably the oral route; whereas animals that received a mucosal prime are boosted by the parenteral route. The parenteral boosting component is administered subcutaneously, intradermally or intramuscularly, preferably intradermally at a dose of 106 cfu. The mucosal boosting component is administered by a mucosal route of inoculation, preferably the oral at a dose of 10⁴-10⁹ cfu, preferably 10⁶-10⁷ cfu.

Ten weeks after the boost, the animals from each group are aerosol challenged with low-dose Mtb strain Erdman and protection is measured by reduction of bacterial burden at 16 weeks post challenge or with survival as endpoint. Methods for handling and challenging Rhesus macaques are documented elsewhere (Capuano et al., Infect. Immun., 71:5831-5844; 2003).

Antigen-specific immunity is assessed by measuring proliferation and IFNg secretion in lymphocyte stimulation tests. The frequency of IFNg producing lymphocytes is determined by enzyme-linked immunosorbent assay (ELISPOT) using the method of Versteegen et al. (J. Immunol. Methods 111:25-29; 1988) as modified by Miyahira et al. (J. Immunol. Methods 181:45-54; 1995) or intracellular cytokine stain and fluorescence-activated cell sorter (FACS) as described (Chattopadhyay et al., Nature Medicine 11, 1113-1117; 2005; Tritel et al., J. Immunol., 171:2538-47; 2003; Hanekom et al., J. Immunol. Methods., 291:185-95; 2004; Berhanu et al., J. Immunol. Methods 279, 199-207; 2003; DeRosa et al., Nature Medicine 7, 245-248; 2001). Blood samples are drawn at weeks 0, 4, 8, 12, 16, 20, and 24 weeks relative to primary vaccination.

Ten weeks after the last immunization the animals are challenged by intratracheal installation of M. tuberculosis strain Erdman (in 3 ml PBS containing 1,000 cfu). All animals are challenged on the same day and with the same preparation. The course of the infection is assessed by monitoring weight, rectal temperature and ESR. Chest x-rays will be performed to detect abnormalities consistent with pulmonary TB at monthly intervals after the challenge, and finally, necropsy at 26 weeks post challenge.

### Clinical Evaluation of TB Vectors and Vaccines

### i) Safety and Toxicity Studies

Preclinical safety and toxicity studies, as mandated by US Food and Drug Association guidelines and CFR21, are performed as described above. Following these studies human safety studies are performed. These studies are performed initially in healthy TB-negative adults, followed by age de-escalation into children and neonates.

Parenteral vaccination of humans with the parenteral component of the present invention is achieved by injecting 1 ml of the vaccine containing 3×10⁴ to 3×10⁷ cfu, preferably 1×10⁵ to 1×10⁶ of the attenuated Mycobacterium strain, such as attenuated Mtb, BCG or rBCG, subcutaneously.

Oral vaccination of humans with the mucosal component of the present invention can be achieved using methods previously described (Miller et al., Can Med Assoc J. 121(1):45-54; 1979). The amount of the live attenuated Mycobacterium strain of the present invention administered orally varies depending on the species of the subject, as well as the disease or condition that is being treated. Generally, the dosage employed is about 10³ to 10¹¹ viable organisms, preferably about 10⁵ to 10⁹ viable organisms.

### EXAMPLES

### Example 1

### Construction of an rBCG Strain that Over-Expresses TB Antigens and Escapes the Endosome

To create a strain that escapes the endosome, we developed BCG1331 derivatives that express perfringolysin O (Pfo), a cytolysin normally secreted by Clostridium perfringens and encoded by the pfoA gene (GenBank Accession no. CPE0163). PfoA mediates escape from phagosome, both in Clostridium and when expressed by B. subtilis (52). Unlike Llo, however, PfoA is active at both pH 5.0 and pH 7.0 (52). To limit to cytotoxicity of Pfo, a mutant form of this protein harboring a G137Q substitution (PfoAG137Q) was utilized as this variant has a short half-life in the host cell cytosol, yet is able to mediate endosome escape over a wide pH range (52).

To explore the utility of PfoAG137Q we constructed an rBCG that secretes this protein, designated AFV102 (i.e. BCG1331 ureC::pfoAG137Q). This strain was constructed by allelic exchange with the ureC gene. As a result, the pfoAG137Q gene expression cassette under the control of the Rv1886c promoter replaced ureC, allowing stable chromosomal expression of PfoA. The allelic exchange plasmid, designated pAF102 (FIG. 2), is composed of the following DNA segments: 1) an oriE sequence for the plasmid to replicate in E. coli; 2) a kanamycin resistant gene sequence for selection in both E. coli and the target BCG strain;

3) the sequences flanking ureC 1 kb upstream (L-flank) and 1 kb downstream (R-flank); and
4) pfoAG137Q under control of the Rv1886c promoter inserted between the ureC flanking sequences gene. Note that the Rv1886 leader peptide sequence was used in place of the wild-type PfoA signal sequence for the secretion of PfoA by rBCG. All these components were synthesized and assembled by Picoscript Inc (Houston, Tex.), resulting in plasmid pAF102 (FIG. 2). The sequence of pAF102 was confirmed by automated dideoxynucleotide sequencing, as described above.

To prepare the target strain, BCG Danish 1331 (BCG1331) was cultured in 7H9 medium supplemented with 10% w/v oleic acid-albumin-dextrose-catalase (OADC; BD Gibco) and 0.05% (v/v) of Tyloxapol (Research and Diagnostic Lab Inc.). When the culture reached log-phase (Optical Density at 550 nm=4-5) the bacteria were collected and prepared for electroporation, as described previously (Sun et al., Mol. Microbiol. 52:25-38; 2004). To generate merodiploids, five micrograms of purified pAF102 DNA was introduced into freshly prepared electrocompetent BCG1331 cells using standard methodologies (Sun et al., 2004). After electroporation the cells were cultured overnight in 7H9 medium supplemented with 10% (v/v) OADC and 0.05% (v/v) of Tyloxapol, then the cells were cultured at 37° C. in 5% v/v CO₂ for 30 days on 7H10 plates supplemented with 50 mcg/ml kanamycin. The resulting merodiploid colonies were transferred to 7H9 medium containing 10% (w/v) sucrose (Sigma, St Louis Mo.) and incubated at 37° C. in 5% v/v CO₂ for an additional 30 days.

One of the colonies, designated AFV102, which arose on the sucrose plates, was found to be urease negative, suggesting that the ureC gene had been replaced by the PfoA expression cassette. The following tests were conducted to verify that strain AFV102 is UreC-negative and PfoA-positive.

First, strain AFV102 was screened for the lack of urease activity using a urease testing kit according to the manufacture's instructions (Difco). Briefly, a 1 mm loop-full of AFV102 bacteria (ca. 110 cfu) was resuspended in the manufacture supplied test buffer in a transparent tube. A similar amount of BCG1331 was used as a urease positive control and a tube containing buffer alone was used as the negative control. The reaction mixture was incubated at room temperature for 30 minutes and the result was judged based on the manufacture's instruction. This assay showed that AFV102 is devoid of urease activity and confirmed the hypothesis that the PfoA expression cassette had replaced this allele

Second, PCR was used to verify the ureC::pfoA genotype in AVF102. PCR forward primer [acggctaccgtctggacat] (SEQ ID NO: 1) and reverse primer [cgatggcttcttcgatgc] (SEQ ID NO: 2) were used at 200 mM in a standard PCR assay to amplify the pfoA allele by initiating the PCR within sequences flanking the ureC gene. The PCR parameters were as follows: Step 1: 95° C. 4 minutes one cycle; Step 2: 95° C. one minute, 60° C. 1 minute, and then 72° C. one minute for a total 30 cycles; Step 3: 72° C. 10 minutes with one cycle. Step 4: 4° C. storage. The resultant PCR products were analyzed by agarose gel electrophoresis and sequenced by automated dideoxynucleotide sequencing techniques, which confirmed the presence of a full-length pfoA gene in place of the ureC gene (i.e. ureC::pfoA) in AFV102. Thus, the PCR results showed that AFV102 produces a DNA band of the expected molecular weight for recombinant ureC::pfoA allele of 2180 bp, while parental strain BCG1331 produces a DNA band of 1967 bp under the same PCR conditions. The PCR product AFV102 was gel purified and sequenced by the commercial sequencing facility of Johns Hopkins University (Baltimore, Md.). The sequencing result confirmed the presence of the recombinant ureC::pfoA allele. In addition, PCR targeted to amplify the kanamycin-resistance gene and the sacB gene from the AFV102 strain failed to produce a PCR product (data not shown), indicating that AFV102 has undergone the final allelic exchange step.

To assess the secretion of PfoAG137Q, AFV102 and BCG1331 were grown to mid-logarithmic phase as described above and the culture supernatants were collected following removal of the bacteria by centrifugation. To test PfoA activity in the supernatants difference dilutions of the culture supernatants in a volume of 100 mcl were combined in 96-well plate with 100 mcl 1% (v/v) sheep erythrocytes and mixed gently. The plates were incubated at 37° C. for 1 h with agitation. To generate a standard curve, serial dilutions of a-hemolysin (Sigma) with known units of hemolysin activity added to sheep erythrocytes in the same plate and incubated as above. At the end of incubation, the plates were subjected to centrifugation at 500×g for 15 min. The supernatants from the V-bottom plate were transferred into equivalent locations in a sterile flat-bottom 96-well plate and the optical density was measured (absorbance at 450 nm minus the absorbance at 540 nm). The hemolytic activity of the PfoA molecule was quantified by measuring the optical density and the intensity of the color measured is proportional to the amount of red cell lysis, which is then in proportion to the quantity of hemolysin. The hemolytic units were calculated in sample values by interpolation using the standard curve. Hemolytic units were defined as the dilution of the sample at which 50% of the sheep red blood cells were lysed. The results of this assay showed that AFV102 produces between 2-10 units of hemolysin activity per 105 bacteria. Taken together with the PCR data, we concluded that AFV102 harbors the pfoA gene in the ureC locus and is capable of secreting PfoA as a function hemolysin.

Furthermore, fluorescent microscopy revealed that PfoAG137Q enables rBCG strain AFV102 to escape the endosome. First, the growth of the rBCG strain AFV102 in situ was tested in J774A.1 macrophage-like cells by determining mycobacterial colony-forming units (cfu) in the infected macrophages, infected at a multiplicity of infection of ten AFV102 cfu to one J774.1 macrophage, as described (Sun et al., 2004). The efficacy of phagocytosis was determined by testing the intracellular cfu counts 3 hr after infection of J774A.1 cells. Subsequent long-term intracellular survival was performed by lysis of the cells to release the intracellular bacteria for enumeration, as previously described (Sun et al., 2004). The results show that AFV102 and BCG1331 display indistinguishable uptake and survival in J774.1 cells, indicating that expression of PfoAG137Q does not significantly alter short-term intracellular survival in macrophages.

In addition, the cytotoxicity of the recombinant strain on J774A.1 macrophages (ATCC No. A TIB-67) was determined by measuring the Lactate Dehydrogenase (LDH) released from infected cells using a “Cell Titer 96 Aqueous One Solution Cell Proliferation Assay” kit (Promega, cat #: G3580) according to the manufacture's instruction. Thus, at different times post infection of J774.1 cells supernatants were measured for the amount of LDH released. Uninfected normal cells were used as a negative control. The percentage of viable cells was calculated based on the amount of LDH released from the infected cells to that of the negative control cells (100% cell viability). The results showed that cytotoxicity of AFV102 is indistinguishable from that of BCG1331.

Finally, fluorescent microscopy was used to determine the intracellular compartment in which AFV102 resides, using similar methods to those previously described (Armstrong and Hart, J. Exp. Med., 134:713-40; 1971; Hasan et al., Mol Microbiol 25:427; 1997; Via et al., J Biol Chem., 272:13326-13331; 1997; Sun et al., Mol. Microbiol. 52:25-38; 2004). Before infection, the bacterial cells were labeled with Alexa Fluor 568 succinimidyl ester (Molecular Probes, Eugene, Oreg.) in PBS at room temperature for 1-1.5 hours according to the manufacture's instruction. This dye forms stable amide bonds to the primary amines located on the bacterial surface. Thus, 10 ml of a AFV102 and BCG1331 cultures were pelleted and resuspended in 25 mls of 0.625 ug/ml of Alexa Fluor 568 in PBS (pH7.2) and incubated at room temperature for 1-1.5 hours to label the bacteria. The labeled bacterial cells were then washed three times with PBS and resuspended in 7H9 growth medium and stored in the refrigerator overnight. J774A.1 cells were cultured in DMEM medium as previously described (Sun et al, 2004) in 6 well cell culture plates on human fibronectin coated coverslips. The cells were plated at a density of 3×106 cells/well and cultured for 2 days in a 37° C. incubator with 5% CO2 and humidity. During the infection, the labeled bacteria were pelleted and resuspended in DMEM+10% FBS medium and added directly to J774A.1 cells with a multiplicity of infection (MOI) of 10 for each cell. After 20 min, 8 hours and 24 hours, the cells were washed with room temperature (RT) phosphate buffered saline (PBS, pH 7.2). The cells were then fixed for 20 minutes at RT with 2% paraformaldehyde in PBS (pH 7.2). The fixed cells were then permeabilized with 0.1% Triton X-100 in PBS (pH 7.2) for 10 minutes at RT followed by washing twice with PBS (pH 7.2). Blocking was done for at least 2 hours at RT or overnight at 4° C. with 3% bovine serum albumin (BSA), 5% normal goat serum (NGS), and 0.5% sodium azide in PBS (pH7.2). Blocking buffer was removed and then rat anti-mouse transferring receptor-FITC (US Biological, Swampscott, Mass.) was added at a dilution of 1:50 in PBS (pH 7.2) containing 1% BSA, 3% NGS, and 0.5% sodium azide followed by incubation at RT for at least 1 hour. Cells were then washed 2-3 times with PBS and mounted with vectsheild mounting media on glass slides. Analysis was done at a magnification of 1500 using a Nikon TE2000 inverted microscope equipped with a Retiga EXI Mono, 12 bit cooled, IR filtered digital camera for imaging.

The results showed that 75% of AFV102 bacilli were observed outside the endosome 24 hr after infection of J744.1 macrophages (ATCC no. TIB-67), whereas only a minority of BCG bacilli appeared in an extra-endosomal compartment. These data suggest that AFV102 and derivatives of this strain will induce stronger CD8+ T cell responses, compared to those induced by BCG and rBCG-Llo+(Hess et al., Proc Natl Acad. Sci., 95:5299-5304; 1998; Grode et al., Clin Invest., 115:2472-2479; 2005).

To over-express TB antigens into rBCG strain AFV102, sequences encoding the Rv3031 promoter functionally linked to sequences encoding Rv3804c (also known as Ag85A), Rv1886 (also known as Ag85B) and Rv0288 (also known as TB10.4) were inserted into the PacI site of pAF100. The resulting plasmid, pAF105 (FIG. 3), was subsequently digested with restriction endonuclease NdeI to remove the E. coli replicon and kanamycin-resistance gene, and re-circularized by ligation with T4 ligase. This DNA (1-2 mg) was introduced into rBCG strain AFV102 by electroporation. The bacteria were cultured in 8.75 cm plates containing 25-30 ml of solid media (Middlebrook 7H10). Following a prescreen by PCR to detect colonies which harbor the antigen expression plasmid, a selected rBCG colony, which is both PfoA-positive and contains the TB antigen expression cassette, is designated AFRO-1 and is expanded to 500 ml in agitated liquid media (Middlebrook 7H9) at 37° C. Once the culture reaches late-log phase, glycerol is added to the 500 ml culture to a final concentration of 10% (v/v) and the premaster seed is stored in 5 ml aliquots at −80° C.

The purity of BCG and rBCG cultures are evaluated by evenly spreading 100 ml aliquots of the BCG culture serially diluted (e.g. 10-fold steps from Neat-10-8) in phosphate buffered saline (PBS) onto 8.75 cm plates containing 25-30 ml of solid media (Middlebrook 7H10). PCR and restriction endonuclease analysis of plasmid DNA is used to confirm that the desired genotype is present in each rBCG isolate. In addition, PCR-generated DNA fragments are sequenced by automated dideoxynucleotide sequencing techniques to confirm the presence of full-length genes.

To assess the secretion of PfoA by AFV102 and AFRO-1 harboring the TB antigen expression plasmid, both strains are grown to mid-logarithmic phase, as described above. The culture supernatants of these cultures are collected and filtered through 0.2-mm membrane filters, as previously described (Hess et al., Proc. Natl. Acad. Sci., 95:5299-304; 1998). The culture filtrate proteins then are assessed for hemolytic activity, as described above. The results show that AFV102 and AFRO-1 display similar levels of hemolytic activity and that AFRO-1 retains the ureC::pfoAG137Q allele and expresses a functional PfoA protein.

Finally, expression of the TB antigens is assessed in culture supernatants proteins separated on 10-15% SDS-PAGE gels. The results show increased expression of Rv3804c and Rv1886. Since Rv0288 is not expected to be over expressed in the culture supernatant, over expression of this 10 kDa protein, which is expressed on the same mRNA as Rv3804c and Rv1886, is inferred by the observation that Rv3804c and Rv1886 are over expressed. Taken as a whole, this example demonstrates that it is possible to generate and rBCG strain which both expresses PfoA and over expresses TB antigens. Such as strain has potential to serve as a second generation TB vaccine.

### Example 2

### Optimization of an Oral rBCG Vaccine Formulation and Dose

Prior to testing the novel two-component TB vaccine a study is conducted to determine the optimal oral formulation and dose. Groups of 16 BALB/c mice are inoculated by gastric intubation as shown in Table 5. 72 hr after vaccination, 3 mice in each group are sacrificed and the numbers of viable AFV102 bacilli in the intestines, Peyer's patches, lungs and spleen are enumerated by direct plate count as above. This experiment shows that oral formulations containing CeraVacx, which includes a stomach neutralizing components, are more effective at enabling the delivery of viable organisms to the mucosal tissues than those without.

Six weeks after vaccination 5 animals in each group are sacrificed and the magnitude of the immune response to Rv3804c, Rv1886 and Rv0288 are measured by flow cytometry. Briefly, the mice are sacrificed by cervical dislocation and spleens are collected under sterile conditions by carefully removing adhesive lipid tissue with sterile tweezers. After rinsing spleens in a 15 mL conical tube containing 10 mL complete RPMI media (R10; RPMI 1640 containing 10% FBS (HyClone), 55 μM 2-Mercaptoethanol, 10 mM HEPES, 2 mM L-glutamine and 1× penicillin-streptomycin solution (all from Gibco), single cell suspensions are prepared by pressing the spleens through 70 μm cell strainers (Falcon). Cells are resuspended in 15 ml incomplete RPMI and centrifuged for 5 minutes at 520 rcf at 4° C. Remaining erythrocytes are lysed with 1 ml ACK lysis buffer (BioWhittacker) per spleen for 2 minutes at room temperature. Following another washing step with 9 mL of R10 medium cells are resuspended in 3 ml R10 and filtered again through a 70 μm cell strainer into a new 15 ml tube. Cells are counted and resuspended at 15×106 cells/ml in R10 medium.

Stimulation for assessment of cytokine production is performed as follows: Dimethyl Sulfoxide (DMSO, Sigma) as negative control and peptide pools are pre-diluted in R10 medium (Life Technologies) containing 1 μg/mL CD28 and CD49d. Final concentration is 2 μg peptide/ml. Phorbol-12-myristate-13-acetate (0.1 μg/ml)/Ionomycin (4 μg/ml) (PMA/I; both purchased from Sigma) served as positive control. After placing 100 μl of the solutions in appropriate wells of 96-well round bottom cell culture plates, 100 μl cell suspension is added and incubated for 1 hour at 37° C. and 5% CO₂. After the addition of 25 μl Golgi-Plug (1:25 diluted into R10 medium), plates are incubated for additional 4-5 hours. Following incubation, plates are stored at 4° C. over night until processing for the intracellular cytokine stain. Plates are spun at 350×g for 3 minutes at 4° C. Supernatants are discarded, cells resuspended and washed with 100 μl of PBS/well at 350×g for 3 minutes at 4° C. After discarding the supernatants and resuspending the cells by carefully vortexing the plate, 50 μL of PBF (i.e. PBS+0.5% FBS) containing 1 μl FcR Block (BD) is added to all wells and incubated for 10 minutes on ice. 150 μl of PBF is added to each well and cells are washed as above. Cells are stained with either pre-titrated Anti-CD4-PC5 or Anti-CD8-PC5 (BD) antibodies in 50 μl PBF buffer for 30 min at 4° C. in the dark. Following the incubation, cells are washed twice with 150 μl PBF buffer. For permeabilization of cells, 100 μl of Cytofix/Cytoperm buffer (BD) is added to each sample well and incubated for 20 minutes at 4° C. in the dark. After that, plates are spun and cells washed with 150 ml Perm/Wash buffer (BD). For intracellular cytokine staining, anti-IFNγ Alexa Fluor 488, anti-TNFα-PE and anti-IL-2-APC (all BD) are pre-diluted 1:40 in Perm/Wash buffer and 50 ml is added per well and incubated for 30 minutes at 4° C. in the dark. Following the incubation, cells are washed twice with 150 μl 1× Perm/Wash Buffer and spun at 350×g for 3 minutes at 4° C. After discarding the supernatant, cells are fixed by adding 220 ml of 1% formaldehyde (Sigma) in PBS. For analysis, 100,000 target cell events are collected from each sample on a CyFlow ML (Partec, Germany) flow cytometer. All sample analysis is performed with FlowJo software (TreeStar Inc., USA) and statistics are determined by using Prism software (GraphPad, USA.).

The remaining 8 animals in each group are challenge 10 weeks after vaccination with Mtb Erdman by an aerosol generated from a 10-ml single-cell suspension containing a total of 107 cfu of the challenge strain. This results in a dose of 100 live bacteria to the lungs of each animal (Turner et al., Infect. Immun., 68:3674-3679; 2000; McMurray et al., Infect. Immun. 50:555-559; 1985; Wiegeshaus et al., Am. Rev. Respir. Dis., 102:422-429; 1970). Following the challenge, the animals are monitored for survival along with unchallenged control animals. The animals are also monitored for weight loss and general health.

Five weeks after challenge, the animals in each group are sacrificed for histopathology and microbiology analysis. Lung and spleen tissues from the mice are evaluated for cfu counts. Since Mtb Erdman strain is used to challenge, TCH is added to the media to distinguish vaccine strain, which is sensitive to TCH, from the challenge strain. The results of this experiment demonstrate that the oral formulations containing CeraVacx are effective at providing protection against an Mtb challenge.

After successful completion of this study, the vaccine which induces similar or better immune responses and protection to Mtb with the fewest vaccine organisms is selected as the optimum formulation and dose.

TABLE 5
Experimental design

| Group | Vaccine formulation | Dose |
|---|---|---|
| 1 | AFRO-1 administered orally in 10% glycerol | 10⁹ |
| 2 | AFRO-1 administered orally in 10% glycerol + 0.2 ml | 10⁹ |
| | CeraVacx | |
| 3 | AFRO-1 administered orally in 10% glycerol + 0.2 ml | 10⁸ |
| | CeraVacx | |
| 4 | AFRO-1 administered orally in 10% glycerol + 0.2 ml | 10⁷ |
| | CeraVacx | |
| 5 | AFRO-1 administered orally in 10% glycerol + 0.2 ml | 10⁵ |
| | CeraVacx | |
| 6 | AFRO-1 administered orally in 10% glycerol + 0.2 ml | 10⁴ |
| | CeraVacx | |
| 7 | AFRO-1 administered orally in 10% glycerol + 0.2 ml | 10³ |
| | CeraVacx | |
| 8 | AFRO-1 (subcutaneously) | 3 × 10⁵ |

### Example 3

### Optimization of Vaccination Regimen

The goal of this experiment is to optimize the prime-boost regimen of candidate attenuated Mycobacterium vaccine strain AFRO-1 (Example 1) in SPF male Hartley guinea pigs (250-300 grams). Accordingly, groups of 10 animals are immunized in as shown in Table 6 so as to evaluate 10, 14 and 17 week prime-boost intervals.

TABLE 6
Guinea pig regimen study design

| | Prime I | Prime II | Prime III | Boost |
|---|---|---|---|---|
| Group | (Day 1) | (Week 3) | (Week 7) | (Week 17) |
| 1 | Saline (id) | — | — | |
| 3 | AFRO-1 (id) | — | — | AFRO-1 (po) |
| 4 | — | AFRO-1 (id) | — | AFRO-1 (po) |
| 5 | — | — | AFRO-1 (id) | AFRO-1 (po) |
| 6 | — | — | — | AFRO-1 (po) |

The primes are administered intradermally at a dose of 10⁶ cfu in 0.1 ml of 10% glycerol. Control mice are given 0.1 ml 10% glycerol intradermally alone. At 14 weeks after the prime the guinea pigs are boosted with the boosting component of the two-component TB vaccine. In group 5 the boost is administered intradermally at a dose of 10⁶ cfu in 0.1 ml of 10% glycerol. In groups 4 and 6 the boosts are administered by intragastric intubation at a dose of 10⁷ cfu suspended in 0.5 ml of 10% (v/v) glycerol and 50% (v/v) CeraVacx.

At 10 weeks after the final immunization, the animals are challenged by aerosol with Mtb strain Erdman by an aerosol generated from a 10-ml single-cell suspension containing a total of 10⁷ cfu of Mtb; this procedure delivers ˜100 live bacteria to the lungs of each animal, as described previously (Brodin et al., 2004). At 5 weeks after the challenge, the animals in each group are sacrificed and the lungs and spleens are collected for histological and microbiological analysis. In the latter instance, lung and spleen tissues from the guinea pigs are evaluated for cfu counts. Since Mtb Erdman strain is used to challenge, TCH is added to the media to distinguish vaccine strain, which is sensitive to TCH, from the challenge strain.

The results of this study identify the optimal interval between the priming and boosting components of the two-component TB vaccine.

### Example 4

### Induction of Protection in Guinea Pigs

To measure the potency of candidate attenuated Mycobacterium vaccine strain AFRO-1 (Example 1) against Mtb challenge, groups of 8 (young-adult SPF Hartley guinea pigs (250-300 grams) are immunized, with priming component of the two-component TB vaccine, BCG or saline as shown in Table 7.

TABLE 7
Guinea pig challenge study design

| | Prime | Boost | Challenge |
|---|---|---|---|
| Group | (Day 1) | (Week 14) | (Week 28) |
| 1 | Saline (id) | CeraVacx | 100 cfu Erdman |
| 3 | AFRO-1 (id) | CeraVacx | 100 cfu Erdman |
| 4 | Saline (id) | AFRO-1 (po) | 100 cfu Erdman |
| 5 | AFRO-1 (id) | AFRO-1 (id) | 100 cfu Erdman |
| 6 | AFRO-1 (id) | AFRO-1 (po) | 100 cfu Erdman |

The primes are administered intradermally at a dose of 10⁶ cfu in 0.1 ml of 10% glycerol. Control mice are given 0.1 ml 10% glycerol intradermally alone. At 14 weeks after the prime the guinea pigs are boosted with the boosting component of the two-component TB vaccine. In group 5 the boost is administered intradermally at a dose of 10⁶ cfu in 0.1 ml of 10% glycerol. In groups 4 and 6 the boosts are administered by intragastric intubation at a dose of 10⁷ cfu suspended in 0.5 ml of 10% (v/v) glycerol and 50% (v/v) CeraVacx.

At 14 weeks after the final immunization, the animals are challenged by aerosol with the Mtb by an aerosol generated from a 10-ml single-cell suspension containing a total of 10⁷ cfu of Mtb; this procedure delivers ˜100 live bacteria to the lungs of each animal, as described previously (Brodin et al., 2004). Following challenge, the animals are monitored for survival along with a healthy group of unvaccinated, unchallenged animals. The animals are also monitored for weight loss and general health.

The results of this study demonstrate that sham-immunized animals die most rapidly after challenge, animals vaccinated with BCG intradermally without a boost display an intermediate mean time to death and animals immunized with the novel two-component TB vaccine survive the longest.

### Example 5

### Protection in Non-Human Primates

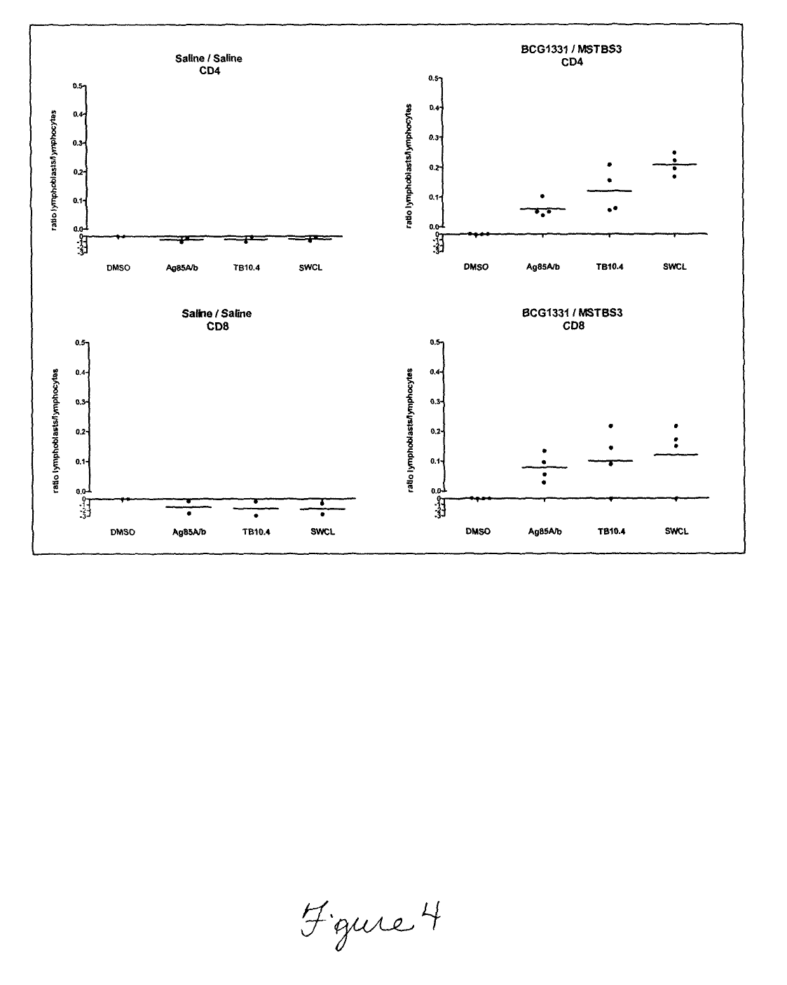

As discussed earlier, the Rhesus macaque serves as a useful model for evaluation of vaccines against Mtb. To demonstrate the utility of a parenteral prime followed by a mucosal (oral) booster vaccine, the immune responses elicited by boosting BCG vaccinated non-human primates with an orogastrically delivered Shigella carrying recombinant nucleocapsids encoding a fusion of Ag85A-Ag85B-TB10.4 (MSTBS3) were evaluated. Rhesus macaques were primed intradermally with 2×105 CFU BCG and boosted intragastrically with 1×1010 CFU of MSTBS3. Heparinized blood was drawn 2 weeks post boost and incubated with specific peptide pools (Ag85A/B and TB10.4) for 7 days. Following the incubation, cells were stained with surface specific antibodies against CD4 and CD8 and fixed in 1% PFA for analysis by flow cytometry. Sample analysis was performed with FlowJo software (TreeStar Inc., USA). Ratios of lymphoblasts to lymphocytes following stimulation were calculated and plotted using Prism software (GraphPad, USA.). The results, presented in FIG. 4, suggest that BCG/MSTBS3-vaccinated monkeys developed specific CD8+ and CD4+ T-cell responses to Ag85A/B and TB10.4 peptide pools and the Shigella Whole Cell Lysate (SWCL), whereas non-vaccinated animals did not develop measurable T-cell responses.

### Example 6

### Comparison of Standard BCG to Two-Component Vaccine

The aim of the study described below is to demonstrate the potency of a standard BCG vaccine vs the two-component TB vaccine of the present invention. The study comprises six groups of 10 animals as shown in Table 8.

TABLE 8
Studies in non-human primates

| Group | Prime | Boost (Week 17) |
|---|---|---|
| 1 | None | None |
| 2 | AFRO-1 (10⁵ cfu →subcutaneous) | AFRO-1 (10⁵ cfu →subcutaneous) |
| 3 | AFRO-1 (10⁵ cfu →oral) | AFRO-1 (10⁵ cfu →subcutaneous) |
| 4 | AFRO-1 (10⁵ cfu →subcutaneous) | AFRO-1 (10⁵ cfu →oral) |
| 5 | Shigella MSTBS3 (10⁵ cfu →oral) | Shigella MSTBS3 (10⁵ cfu →oral) |
| 6 | AFRO-1 (10⁵ cfu →subcutaneous) | Shigella MSTBS3 (10⁵ cfu →oral) |

Formulation of the oral priming component is described elsewhere (Adwell et al., Vaccine, 22:70-76; 2003; Buddle et al., Vaccine, 23:3581-3589; 2005). The boost is administered 17 weeks after the prime. The parenteral boosting component is administered subcutaneously, intradermally or intramuscularly, preferably intradermally at a dose of 10⁶ cfu. The mucosal boosting component is administered by a mucosal route of inoculation, preferably the oral at a dose of 10⁴-10⁹ cfu, preferably 10⁶-10⁷ cfu.

Ten weeks after the boost, the animals from each group are aerosol challenged with low-dose M. tuberculosis strain Erdman and protection is measured by reduction of bacterial burden at 16 weeks post challenge or with survival as endpoint. Methods for handling and challenging Rhesus macaques are documented elsewhere (Capuano et al., Infect. Immun., 71:5831-5844; 2003).

Ten weeks after the last immunization the animals are challenged by intratracheal installation of M. tuberculosis strain Erdman (in 3 ml PBS containing 1,000 cfu). All animals are challenged on the same day and with the same preparation. The course of the infection is assessed by monitoring weight, rectal temperature and ESR. Chest x-rays will be performed to detect abnormalities consistent with pulmonary TB at monthly intervals after the challenge, and finally, necropsy at 26 weeks post challenge.

The results of this study demonstrate that sham-immunized animals develop severe pulmonary TB (Group 1), animals vaccinated with BCG intradermally two times (Group 2) display modest reduction in the severity of pulmonary TB and animals immunized with the novel two-component TB vaccine (Group 3) survive the longest.

### REFERENCES

1. 1993. Tuberculosis: a global emergency. World Health Forum 14:438.
2. 2004. WHO and UNAIDS call for joint HIV-tuberculosis action. Bull World Health Organ 82:810.
3. 1993. WHO declares tuberculosis a global emergency. Soz Praventivmed 38:251-2.
4. Arthur, J. W., and M. R. Wilkins. 2004. Using proteomics to mine genome sequences. Proteome Res 3:393-402.
5. Aziz, M. A., and A. Wright. 2005. The World Health Organization/International Union Against Tuberculosis and Lung Disease Global Project on Surveillance for Anti-Tuberculosis Drug Resistance: a model for other infectious diseases. Clin Infect Dis 41 Suppl 4:S258-62.
6. Barbosa, T., S. Arruda, B. D. Fernandes, L. P. Carvalho, S. Cardoso, S. Cunha, M. L. Barreto, S. M. Pereira, L. C. Rodrigues, and M. Barral-Netto. 2003. BCG (Bacille of Calmette-Guerin) revaccination leads to improved in vitro IFN-gamma response to mycobacterial antigen independent of tuberculin sensitization in Brazilian school-age children. Vaccine 21:2152-60.
7. Belyakov, I. M., J. D. Ahlers, B. Y. Brandwein, P. Earl, B. L. Kelsall, B. Moss, W. Strober, and J. A. Berzofsky. 1998. The importance of local mucosal HIV-specific CD8(+) cytotoxic T lymphocytes for resistance to mucosal viral transmission in mice and enhancement of resistance by local administration of L-12. J. Clin. Invest. 102:2072-2081.
8. Belyakov, I. M., M. A. Derby, J. D. Ahlers, B. L. Kelsall, P. Earl, B. Moss, W. Strober, and J. A. Berzofsky. 1998. Mucosal immunization with HIV-1 peptide vaccine induces mucosal and systemic cytotoxic T lymphocytes and protective immunity in mice against intrarectal recombinant HIV-vaccinia challenge. Proc. Natl. Acad. Sci. 95:1709-1714.
9. Belyakov, I. M., Z. Hel, B. Kelsall, V. A. Kuznetsov, J. D. Ahlers, J. Nacsa, D. I. Watkins, T. M. Allen, A. Sette, J. Altman, R. Woodward, P. D. Markham, J. D. Clements, G. Franchini, W. Strober, and J. A. Berzofsky. 2001. Mucosal AIDS vaccine reduces disease and viral load in gut reservoir and blood after mucosal infection of macaques. Nat. Med. 7:1320-1326.
10. Belyakov, I. M., B. Moss, W. Strober, and J. A. Berzofsky. 1999. Mucosal vaccination overcomes the barrier to recombinant vaccinia immunization caused by preexisting poxvirus immunity. Proc Natl Acad Sci USA 96:4512-7.
11. Boyton, R. J. 2005. Infectious lung complications in patients with HIV/AIDS. Curr Opin Pulm Med 11:203-7.
12. Brandt, L., M. Elhay, I. Rosenkrands, E. B. Lindblad, and P. Andersen. 2000. ESAT-6 subunit vaccination against Mycobacterium tuberculosis. Infect Immun 68:791-5.
13. Brandt, L., J. Feino Cunha, A. Weinreich Olsen, B. Chilima, P. Hirsch, R. Appelberg, and P. Andersen. 2002. Failure of the Mycobacterium bovis BCG vaccine: some species of environmental mycobacteria block multiplication of BCG and induction of protective immunity to tuberculosis. Infect Immun 70:672-8.
14. Brandt, L., Y. A. Skeiky, M. R. Alderson, Y. Lobet, W. Dalemans, O. C. Turner, R. J. Basaraba, A. A. Izzo, T. M. Lasco, P. L. Chapman, S. G. Reed, and I. M. Orme. 2004. The protective effect of the Mycobacterium bovis BCG vaccine is increased by coadministration with the Mycobacterium tuberculosis 72-kilodalton fusion polyprotein Mtb72F in M. tuberculosis-infected guinea pigs. Infect Immun 72:6622-32.
15. Chadha, V. K. 2005. Tuberculosis epidemiology in India: a review. Int J Tuberc Lung Dis 9:1072-82.
16. Chintu, C., and P. Mwaba. 2005. Tuberculosis in children with human immunodeficiency virus infection. Int J Tuberc Lung Dis 9:477-84.
17. Cole, S. T. 2002. Comparative mycobacterial genomics as a tool for drug target and antigen discovery. Eur Respir J Suppl 36:78s-86s.
18. Cole, S. T., R. Brosch, J. Parkhill, T. Garnier, C. Churcher, D. Harris, S. V. Gordon, K. Eiglmeier, S. Gas, C. E. Barry, 3rd, F. Tekaia, K. Badcock, D. Basham, D. Brown, T. Chillingworth, R. Connor, R. Davies, K. Devlin, T. Feltwell, S. Gentles, N. Hamlin, S. Holroyd, T. Hornsby, K. Jagels, A. Krogh, J. McLean, S. Moule, L. Murphy, K. Oliver, J. Osborne, M. A. Quail, M. A. Rajandream, J. Rogers, S. Rutter, K. Seeger, J. Skelton, R. Squares, S. Squares, J. E. Sulston, K. Taylor, S. Whitehead, and B. G. Barrell. 1998. Deciphering the biology of Mycobacterium tuberculosis from the complete genome sequence. Nature 393:537-44.
19. de Jonge, M. I., R. Brosch, P. Brodin, C. Demangel, and S. T. Cole. 2005. Tuberculosis: from genome to vaccine. Expert Rev Vaccines 4:541-51.
20. Demangel, C., T. Garnier, I. Rosenkrands, and S. T. Cole. 2005. Differential effects of prior exposure to environmental mycobacteria on vaccination with Mycobacterium bovis BCG or a recombinant BCG strain expressing RD1 antigens. Infect Immun 73:2190-6.
21. Dietrich, J., C. Aagaard, R. Leah, A. W. Olsen, A. Stryhn, T. M. Doherty, and P. Andersen. 2005. Exchanging ESAT6 with TB10.4 in an Ag85B fusion molecule-based tuberculosis subunit vaccine: efficient protection and ESAT6-based sensitive monitoring of vaccine efficacy. J Immunol 174:6332-9.
22. Dobos, K. M., J. S. Spencer, I. M. Orme, and J. T. Belisle. 2004. Proteomic approaches to antigen discovery. Methods Mol Med 94:3-17.
23. Dourado, I., M. H. Rios, S. M. Pereira, S. S. Cunha, M. Y. Ichihara, J. C. Goes, L. C. Rodrigues, A. L. Bierrenbach, and M. L. Barreto. 2003. Rates of adverse reactions to first and second doses of BCG vaccination: results of a large community trial in Brazilian schoolchildren. Int J Tuberc Lung Dis 7:399-402.
24. Dye, C., C. J. Watt, D. M. Bleed, S. M. Hosseini, and M. C. Raviglione. 2005. Evolution of tuberculosis control and prospects for reducing tuberculosis incidence, prevalence, and deaths globally. Jama 293:2767-75.
25. Edwards, K. M., M. H. Cynamon, R. K. Voladri, C. C. Hager, M. S. DeStefano, K. T. Tham, D. L. Lakey, M. R. Bochan, and D. S. Kernodle. 2001. Iron-cofactored superoxide dismutase inhibits host responses to Mycobacterium tuberculosis. Am J Respir Crit. Care Med 164:2213-9.
26. Elzing a, G., M. C. Raviglione, and D. Maher. 2004. Scale up: meeting targets in global tuberculosis control. Lancet 363:814-9.
27. Gentschev, I., Z. Sokolovic, H. J. Mollenkopf, J. Hess, S. H. Kaufmann, M. Kuhn, G. F. Krohne, and W. Goebel. 1995. Salmonella strain secreting active listeriolysin changes its intracellular localization. Infect. Immun. 63:4202-4205.
28. Goonetilleke, N. P., H. McShane, C. M. Hannan, R. J. Anderson, R. H. Brookes, and A. V. Hill. 2003. Enhanced immunogenicity and protective efficacy against Mycobacterium tuberculosis of bacille Calmette-Guerin vaccine using mucosal administration and boosting with a recombinant modified vaccinia virus Ankara. J Immunol 171:1602-9.
29. Grode, L., M. Kursar, J. Fensterle, S. H. Kaufmann, and J. Hess. 2002. Cell-mediated immunity induced by recombinant Mycobacterium bovis Bacille Calmette-Guerin strains against an intracellular bacterial pathogen: importance of antigen secretion or membrane-targeted antigen display as lipoprotein for vaccine efficacy. J Immunol 168:1869-76.
30. Herremans, T. M., J. H. Reimerink, A. M. Buisman, T. G. Kimman, and M. P. Koopmans. 1999. Induction of mucosal immunity by inactivated poliovirus vaccine is dependent on previous mucosal contact with live virus. J. Immunol. 162:5011-5018.
31. Hess, J., D. Miko, A. Catic, V. Lehmensiek, D. G. Russell, and S. H. Kaufmann. 1998. Mycobacterium bovis Bacille Calmette-Guerin strains secreting listeriolysin of Listeria monocytogenes. Proc Natl Acad Sci USA 95:5299-304.
32. Hondalus, M. K., S. Bardarov, R. Russell, J. Chan, W. R. Jacobs, Jr., and B. R. Bloom. 2000. Attenuation of and protection induced by a leucine auxotroph of Mycobacterium tuberculosis. Infect Immun 68:2888-98.
33. Horwitz, M. A., and G. Harth. 2003. A new vaccine against tuberculosis affords greater survival after challenge than the current vaccine in the guinea pig model of pulmonary tuberculosis. Infect Immun 71:1672-9.
34. Horwitz, M. A., G. Harth, B. J. Dillon, and S. Maslesa-Galic. 2005. Enhancing the protective efficacy of Mycobacterium bovis BCG vaccination against tuberculosis by boosting with the Mycobacterium tuberculosis major secretory protein. Infect Immun 73:4676-83.
35. Horwitz, M. A., G. Harth, B. J. Dillon, and S. Maslesa-Galic. 2000. Recombinant bacillus calmette-guerin (BCG) vaccines expressing the Mycobacterium tuberculosis 30-kDa major secretory protein induce greater protective immunity against tuberculosis than conventional BCG vaccines in a highly susceptible animal model. Proc Natl Acad Sci USA 97:13853-8.
36. Horwitz, M. A., B. W. Lee, B. J. Dillon, and G. Harth. 1995. Protective immunity against tuberculosis induced by vaccination with major extracellular proteins of Mycobacterium tuberculosis. Proc Natl Acad Sci USA 92:1530-4.
37. Huygen, K., J. Content, O. Denis, D. L. Montgomery, A. M. Yawman, R. R. Deck, C. M. DeWitt, I. M. Orme, S. Baldwin, C. D'Souza, A. Drowart, E. Lozes, P. Vandenbussche, J. P. Van Vooren, M. A. Liu, and J. B. Ulmer. 1996. Immunogenicity and protective efficacy of a tuberculosis DNA vaccine. Nat Med 2:893-8.
38. Irwin, S. M., A. A. Izzo, S. W. Dow, Y. A. Skeiky, S. G. Reed, M. R. Alderson, and I. M. Orme. 2005. Tracking antigen-specific CD8 T lymphocytes in the lungs of mice vaccinated with the Mtb72F polyprotein. Infect Immun 73:5809-16.
39. Kantele, A., M. Hakkinen, Z. Moldoveanu, A. Lu, E. Savilahti, R. D. Alvarez, S. Michalek, and J. Mestecky. 1998. Differences in immune responses induced by oral and rectal immunizations with Salmonella typhi Ty21a: evidence for compartmentalization within the common mucosal immune system in humans. Infect. Immun. 66:5630-5635.
40. Kita, Y., T. Tanaka, S. Yoshida, N. Ohara, Y. Kaneda, S. Kuwayama, Y. Muraki, N. Kanamaru, S. Hashimoto, H. Takai, C. Okada, Y. Fukunaga, Y. Sakaguchi, I. Furukawa, K. Yamada, Y. Inoue, Y. Takemoto, M. Naito, T. Yamada, M. Matsumoto, D. N. McMurray, E. C. Cruz, E. V. Tan, R. M. Abalos, J. A. Burgos, R. Gelber, Y. Skeiky, S. Reed, M. Sakatani, and M. Okada. 2005. Novel recombinant BCG and DNA-vaccination against tuberculosis in a cynomolgus monkey model. Vaccine 23:2132-5.
41. Kutteh, W. H., A. Kantele, Z. Moldoveanu, P. A. Crowley-Nowick, and J. Mestecky. 2001. Induction of specific immune responses in the genital tract of women after oral or rectal immunization and rectal boosting with Salmonella typhi Ty21a vaccine. J Reprod Immunol 52:61-75.
42. Langermans, J. A., T. M. Doherty, R. A. Vervenne, T. van der Laan, K. Lyashchenko, R. Greenwald, E. M. Agger, C. Aagaard, H. Weiler, D. van Soolingen, W. Dalemans, A. W. Thomas, and P. Andersen. 2005. Protection of macaques against Mycobacterium tuberculosis infection by a subunit vaccine based on a fusion protein of antigen 85B and ESAT-6. Vaccine 23:2740-50.
43. Maher, D., A. Harries, and H. Getahun. 2005. Tuberculosis and HIV interaction in sub-Saharan Africa: impact on patients and programmes; implications for policies. Trop Med Int Health 10:734-42.
44. Maher, D., and M. Raviglione. 2005. Global epidemiology of tuberculosis. Clin Chest Med 26:167-82, v.
45. McShane, H., A. A. Pathan, C. R. Sander, N. P. Goonetilleke, H. A. Fletcher, and A. V. Hill. 2005. Boosting BCG with MVA85A: the first candidate subunit vaccine for tuberculosis in clinical trials. Tuberculosis (Edinb) 85:47-52.
46. McShane, H., A. A. Pathan, C. R. Sander, S. M. Keating, S.C. Gilbert, K. Huygen, H. A. Fletcher, and A. V. Hill. 2004. Recombinant modified vaccinia virus Ankara expressing antigen 85A boosts BCG-primed and naturally acquired antimycobacterial immunity in humans. Nat Med 10: 1240-4.
47. Olsen, A. W., P. R. Hansen, A. Holm, and P. Andersen. 2000. Efficient protection against Mycobacterium tuberculosis by vaccination with a single subdominant epitope from the ESAT-6 antigen. Eur J Immunol 30:1724-32.
48. Olsen, A. W., A. Williams, L. M. Okkels, G. Hatch, and P. Andersen. 2004. Protective effect of a tuberculosis subunit vaccine based on a fusion of antigen 85B and ESAT-6 in the aerosol guinea pig model. Infect Immun 72:6148-50.
49. Pal, P. G., and M. A. Horwitz. 1992. Immunization with extracellular proteins of Mycobacterium tuberculosis induces cell-mediated immune responses and substantial protective immunity in a guinea pig model of pulmonary tuberculosis. Infect Immun 60:4781-92.
50. Palendira, U., J. M. Spratt, W. J. Britton, and J. A. Triccas. 2005. Expanding the antigenic repertoire of BCG improves protective efficacy against aerosol Mycobacterium tuberculosis infection. Vaccine 23:1680-5.
51. Park, H. D., K. M. Guinn, M. I. Harrell, R. Liao, M. I. Voskuil, M. Tompa, G. K. Schoolnik, and D. R. Sherman. 2003. Rv3133c/dosR is a transcription factor that mediates the hypoxic response of Mycobacterium tuberculosis. Mol Microbiol 48:833-43.
52. Portnoy, D. A., R. K. Tweten, M. Kehoe, and J. Bielecki. 1992. Capacity of listeriolysin O, streptolysin O, and perfringolysin O to mediate growth of Bacillus subtilis within mammalian cells. Infect Immun 60:2710-7.
53. Pym, A. S., P. Brodin, L. Majlessi, R. Brosch, C. Demangel, A. Williams, K. E. Griffiths, G. Marchal, C. Leclerc, and S. T. Cole. 2003. Recombinant BCG exporting ESAT-6 confers enhanced protection against tuberculosis. Nat Med 9:533-9.
54. Rodrigues, L. C., S. M. Pereira, S. S. Cunha, B. Genser, M. Y. Ichihara, S.C. de Brito, M. A. Hijjar, 1. Dourado, A. A. Cruz, C. Sant'Anna, A. L. Bierrenbach, and M. L. Barreto. 2005. Effect of BCG revaccination on incidence of tuberculosis in school-aged children in Brazil: the BCG-REVAC cluster-randomised trial. Lancet 366:1290-5.
55. Russell, M. W., Z. Moldoveanu, P. L. White, G. J. Sibert, J. Mestecky, and S. M. Michalek. 1996. Salivary, nasal, genital, and systemic antibody responses in monkeys immunized intranasally with a bacterial protein antigen and the Cholera toxin B subunit. Infect Immun 64:1272-83.
56. Sambandamurthy, V. K., X. Wang, B. Chen, R. G. Russell, S. Derrick, F. M. Collins, S. L. Morris, and W. R. Jacobs, Jr. 2002. A pantothenate auxotroph of Mycobacterium tuberculosis is highly attenuated and protects mice against tuberculosis. Nat Med 8:1171-4.
57. Sampson, S. L., C. C. Dascher, V. K. Sambandamurthy, R. G. Russell, W. R. Jacobs, Jr., B. R. Bloom, and M. K. Hondalus. 2004. Protection elicited by a double leucine and pantothenate auxotroph of Mycobacterium tuberculosis in guinea pigs. Infect Immun 72:3031-7.
58. Schnappinger, D., S. Ehrt, M. I. Voskuil, Y. Liu, J. A. Mangan, I. M. Monahan, G. Dolganov, B. Efron, P. D. Butcher, C. Nathan, and G. K. Schoolnik. 2003. Transcriptional Adaptation of Mycobacterium tuberculosis within Macrophages: Insights into the Phagosomal Environment. J Exp Med 198:693-704.
59. Sharma, D.C. 2004. Tuberculosis control goals unlikely to be met by 2005. Lancet 363:1122.
60. Skeiky, Y. A., M. R. Alderson, P. J. Ovendale, J. A. Guderian, L. Brandt, D.C. Dillon, A. Campos-Neto, Y. Lobet, W. Dalemans, I. M. Orme, and S. G. Reed. 2004. Differential immune responses and protective efficacy induced by components of a tuberculosis polyprotein vaccine, Mtb72F, delivered as naked DNA or recombinant protein. J Immunol 172:7618-28.
61. Taelman, H., J. Batungwanayo, J. Clerinx, A. Kagame, J. Bogaerts, S. Allen, and P. Van de Perre. 1991. Risk of tuberculosis among women of childbearing age with HIV infection in Zaire. Am Rev Respir Dis 144:1219-20.
62. Tanghe, A., S. D'Souza, V. Rosseels, O. Denis, T. H. Ottenhoff, W. Dalemans, C. Wheeler, and K. Huygen. 2001. Improved immunogenicity and protective efficacy of a tuberculosis DNA vaccine encoding Ag85 by protein boosting. Infect Immun 69:3041-7.
63. Thillagavathie, P. 2000. Current issues in maternal and perinatal tuberculosis: impact of the HIV-1 epidemic. Semin Neonatol 5:189-96.
64. Urquhart, B. L., T. E. Atsalos, D. Roach, D. J. Basseal, B. Bjellqvist, W. L. Britton, and I. Humphery-Smith. 1997. ‘Proteomic contigs’ of Mycobacterium tuberculosis and Mycobacterium bovis (BCG) using novel immobilised pH gradients. Electrophoresis 18:1384-92.
65. Vallejo, J. G., and J. R. Starke. 1992. Tuberculosis and pregnancy. Clin Chest Med 13:693-707.
66. Voskuil, M. I., D. Schnappinger, R. Rutherford, Y. Liu, and G. K. Schoolnik. 2004. Regulation of the Mycobacterium tuberculosis PE/PPE genes. Tuberculosis (Edinb) 84:256-62.
67. Weinrich Olsen, A., L. A. van Pinxteren, L. Meng Okkels, P. Birk Rasmussen, and P. Andersen. 2001. Protection of mice with a tuberculosis subunit vaccine based on a fusion protein of antigen 85b and esat-6. Infect Immun 69:2773-8.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A method of eliciting both a systemic and a mucosal immune response to live, attenuated Mycobacteria or mycobacterial antigens in a host, comprising the steps of
administering parenterally to said host a first antigenic composition comprising said live, attenuated Mycobacteria, or said mycobacterial antigens, or a vector or bacterium harboring nucleic acids coding for said mycobacterial antigens; and
administering mucosally to said host a second antigenic composition comprising said live, attenuated Mycobacteria, or said mycobacterial antigens, or a vector or bacterium harboring nucleic acids coding for said mycobacterial antigens, said second antigenic composition being different from said first antigenic composition;
wherein said steps of administering parenterally and administering mucosally result in the induction in said host of both a systemic and a mucosal immune response to said live, attenuated Mycobacteria or said mycobacterial antigens.

2. The method of claim 1, wherein said live, attenuated Mycobacteria is selected from the group consisting of Mycobacterium bovis, BCG, Mycobacterium avium complex, Mycobacterium kansasii, Mycobacterium malmoense, Mycobacterium simiae, Mycobacterium szulgai, Mycobacterium xenopi, Mycobacterium scrofulaceum, Mycobacterium abscessus, Mycobacterium chelonae, Mycobacterium haemophilum, Mycobacterium ulcerans, and Mycobacterium marinum.

3. The method of claim 1, wherein said bacterium harboring nucleic acids coding for said mycobacterial antigens is a Shigella bacterium.

4. The method of claim 1, wherein said vector coding for said mycobacterial antigens is an adenoviral vector.

5. The method of claim 1, wherein said live, attenuated Mycobacteria comprises DNA encoding a moiety selected from the group consisting of: a foreign immunogen, an endogenous immunogen, an adjuvant, a cytokine, a pro-apoptosis agent, and an overexpressed Mtb antigen.

6. The method of claim 1, wherein said step of administering mucosally is accomplished orally is carried out before said step of administering parenterally.
